SEQUENCE LISTING (13 sequences)

SEQ ID NO: 1 - protein, 418 aa, Pseudomonas fluorescens
  MISC_FEATURE: isocitrate dehydrogenase enzyme
MGYKKIQVPAVGDKITVNADHSLNVPDNPIIPFIEGDGIGVDVSPVMIKVVDAAVEKAYGGKRKISWMEVYAGEKATQVYDQDTWLPQETLDAVKDYVVSIKGPLTTPVGGGIRSLNVALRQQLDLYVCLRPVVWFEGVPSPVKKPGDVDMVIFRENSEDIYAGIEWKAGSPEATKVIKFLKEEMGVTKIRFDQDCGIGIKPVSKEGTKRLVRKALQYVVDNDRKSLTIVHKGNIMKFTEGAFKDWGYEVAKEEFGAELLDGGPWMKFKNPKTGREVVVKDAIADAMLQQILLRPAEYDVIATLNLNGDYLSDALAAEVGGIGIAPGANLSDTVAMFEATHGTAPKYAGKDQVNPGSVILSAEMMLRHLGWTEAADLIIKGTNGAIKAKTVTYDFERLMEGATLVSSSGFGEALIKHM

SEQ ID NO: 2 - DNA, 1257 bp, Pseudomonas fluorescens
  misc_feature: isocitrate dehydrogenase enzyme coding sequence 1.1.1.42
atgggttacaagaagattcaggttccagccgtcggcgacaaaatcaccgtcaacgcagaccattctctcaatgtccctgataacccgatcattcccttcatcgaaggtgacggcattggcgtcgacgtcagccctgtgatgatcaaagtggttgatgctgccgtagagaaagcctacgggggtaagcgcaagatttcctggatggaggtttatgctggcgaaaaagcaactcaggtctatgaccaggacacctggctgccccaggaaaccctggacgcggtcaaggattacgtggtctccatcaaaggcccgctgaccactccggtcggtggcggcatccgttccctcaacgtcgccctgcgccaacagctcgatctctatgtctgccttcgccctgtggtgtggttcgaaggtgtgccgagcccggtgaaaaagcctggcgacgtcgacatggtgatcttccgcgagaactccgaagacatttatgccggtatcgaatggaaagccggctcccctgaggccaccaaggtcatcaaattcctgaaagaagaaatgggcgtcaccaagatccgtttcgaccaggattgcggcatcggcatcaagccggtttccaaagaaggcaccaagcgtctggtgcgcaaggcgctgcaatacgtggtggacaacgaccgcaagtcgctgaccatcgtgcacaagggcaacatcatgaaattcaccgaaggtgccttcaaggactggggctacgaggtggcgaaggaagaattcggcgccgagctgctcgatggcggcccatggatgaaattcaagaacccgaaaaccggccgcgaagtcgtcgtcaaggacgccatcgccgacgccatgctccagcagatcctgctgcgtccggccgaatacgatgtgatcgccaccctcaacctcaacggtgactacctgtccgacgccctggcggcggaagtgggcggtatcggtatcgcgccgggtgccaacctgtccgacaccgtagccatgttcgaggcgacccacggtactgcgccgaaatatgccggcaaggaccaggtcaacccgggttcggtgattttgtcggcggaaatgatgctgcgccacctgggctggaccgaggcggccgacctgatcatcaagggcaccaatggcgccatcaaggccaagaccgtgacctacgacttcgaacgtctgatggaaggcgccacactggtgtcttcttcgggcttcggtgaagcgctgatcaagcacatgtaa

SEQ ID NO: 3 - protein, 475 aa, Synechococcus elongatus PPC794
  MISC_FEATURE: isocitrate dehydrogenase enzyme
MYEKIQPPSEGSKIRFEAGKPIVPDNPIIPFIRGDGTGVDIWPATERVLDAAVAKAYGGQRKITWFKVYAGDEACDLYGTYQYLPEDTLTAIREYGVAIKGPLTTPIGGGIRSLNVALRQIFDLYACVRPCRYYTGTPSPHRTPEQLDVVVYRENTEDIYLGIEWKQGDPTGDRLIKLLNEDFIPNSPSLGKKQIRLDSGIGIKPISKTGSQRLIRRAIEHALRLEGRKRHVTLVHKGNIMKFTEGAFRDWGYELATTEFRTDCVTERESWILANQESKPDLSLEDNARLIEPGYDAMTPEKQAAVVAEVKAVLDSIGATHGNGQWKSKVLVDDRIADSIFQQIQTRPGEYSVLATMNLNGDYISDAAAAVVGGLGMAPGANIGDEAAIFEATHGTAPKHAGLDRINPGSVILSGVMMLEYLGWQEAADLITKGISQAIANREVTYDLARLMEPAVDQPLKCSEFAEAIVKHFDD

SEQ ID NO: 4 - DNA, 1450 bp, Synechococcus elongatus PCC794
  misc_feature: isocitrate dehydrogenase enzyme coding sequesnce 1.1.1.42
agggcaagcttatgtacgagaagattcaaccccctagcgaaggcagcaaaattcgctttgaagccggcaagccgatcgttcccgacaacccgatcattcccttcattcgtggtgacggcactggcgttgatatctggcccgcaactgagcgcgttctcgatgccgctgtcgctaaagcctatggcggtcagcgcaaaatcacttggttcaaagtctacgcgggtgatgaagcctgcgacctctacggcacctaccaatatctgcctgaagatacgctgacagcgatccgcgagtacggcgtggcaatcaaaggcccgctgacgacgccgatcggtggtggcattcgatcgctgaacgtggcgctacggcaaatcttcgatctctatgcctgcgtccgcccctgtcgctactacaccggcacaccctcgccccaccgcacgcccgaacaactcgatgtggtggtctaccgcgaaaacaccgaggatatctacctcggcatcgaatggaagcaaggtgatcccaccggcgatcgcctgatcaagctgctgaacgaggacttcattcccaacagccccagcttgggtaaaaagcaaatccgtttggattccggcattggtattaagccgatcagtaaaacgggtagccagcgtctgattcgtcgtgcgatcgagcatgccctacgcctcgaaggccgcaagcgacatgtcacccttgtccacaagggcaacatcatgaagttcacggaaggtgctttccgggactggggctatgaactggccacgactgagttccgaaccgactgtgtgactgaacgggagagctggattcttgccaaccaagaaagcaagccggatctcagcttggaagacaatgcgcggctcatcgaacctggctacgacgcgatgacgcccgaaaagcaggcagcagtggtggctgaagtgaaagctgtgctcgacagcatcggcgccacccacggcaacggtcagtggaagtctaaggtgctggttgacgatcgcattgctgacagcatcttccagcagattcaaacccgcccgggtgaatactcggtgctggcgacgatgaacctcaatggcgactacatctctgatgcagcggcggcggtggtcggtggcctgggcatggcccccggtgccaacattggcgacgaagcggcgatctttgaagcgacccacggcacagcgcccaagcacgctggcctcgatcgcattaaccccggctcggtcatcctctccggcgtgatgatgctggagtacctaggctggcaagaggctgctgacttgatcaccaagggcatcagccaagcgatcgctaaccgtgaggtcacctacgatctggctcggttgatggaaccggcggttgatcaaccactcaagtgctcggaatttgccgaagccatcgtcaagcatttcgacgattagggatccagcgc

SEQ ID NO: 5 - protein, 1628 aa, Pseudomonas fluorescens
  MISC_FEATURE: glutamate dehydrogenase enzyme
MAFFTAASKADFQHQLQAALAQHISEQALPQVALFAEQFFGIISLDELTQRRLSDLAGCTLSAWRLLERFDHAQPQVRVYNPDYERHGWQSTHTAVEVLHHDLPFLVDSVRTELNRRGYSIHTLQTTVLSVRRGSKGELLEILPKGTTGEGVLHESLMYLEIDRCANAAELNVLSKELEQVLGEVRVAVSDFEPMKAKVQEILTKLDNSAFAVDADEKNEIKSFLEWLVGNHFTFLGYEEFTVVDQADGGHIEYDQNSFLGLTKMLRTGLTNEDRHIEDYAVKYLREPTLLSFAKAAHPSRVHRPAYPDYVSIREIDADGKVIKEHRFMGLYTSSVYGESVRVIPFIRRKVEEIERRSGFQAKAHLGKELAQVLEVLPRDDLFQTPVDELFSTVMSIVQIQERNKIRVFLRKDPYGRFCYCLAYVPRDIYSTEVRQKIQQVLMERLKASDCEFWTFFSESVLARVQLILRVDPKNRIDIDPLQLENEVIQACRSWQDDYAALTVETFGEANGTNVLADFPKGFPAGYRERFAAHSAVVDMQHLLNLSEKKPLAMSFYQPLASGPRELHCKLYHADTPLALSDVLPILENLGLRVLGEFPYRLRHTNGREFWIHDFAFTAAEGLDLDIQQLNDTLQDAFVHIVRGDAENDAFNRLVLTAGLPWRDVALLRAYARYLKQIRLGFDLGYIASTLNNHTDIARELTRLFKTRFYLARKLGSEDLDDKQQRLEQAILTALDDVQVLNEDRILRRYLDLIKATLRTNFYQTDANGQNKSYFSFKFNPHLIPELPKPVPKFEIFVYSPRVEGVHLRFGNVARGGLRWSDREEDFRTEVLGLVKAQQVKNSVIVPVGAKGGFLPRRLPLGGSRDEIAAEGIACYRIFISGLLDITDNLKDGKLVPPANVVRHDDDDPYLVVAADKGTATFSDIANGIAIDYGFWLGDAFASGGSAGYDHKKMGITAKGAWVGVQRHFRERGINVQEDSITVVGVGDMAGDVFGNGLLMSDKLQLVAAFNHLHIFIDPNPNPATSFAERQRMFDLPRSAWSDYDTSIMSEGGGIFSRSAKSIAISPQMKERFDIQADKLTPTELLNALLKAPVDLLWNGGIGTYVKASTESHADVGDKANDALRVNGNELRCKVVGEGGNLGMTQLGRVEFGLNGGGSNTDFIDNAGGVDCSDHEVNIKILLNEVVQAGDMTDKQRNQLLASMTDEVGGLVLGNNYKQTQALSLAARRAYARIAEYKRLMSDLEGRGKLDRAIEFLPTEEQLAERVAEGHGLTRPELSVLISYSKIDLKEQLLGSLVPDDDYLTRDMETAFPPTLVSKFSEAMRRHRLKREIVSTQIANDLVNHMGITFVQRLKESTGMTPANVAGAYVIVRDIFHLPHWFRQIEALDYQVSADVQLELMDELMRLGRRATRWFLRARRNEQNAARDVAHFGPHLKELGLKLDELLSGEIRENWQERYQAYVAAGVPELLARMVAGTTHLYTLLPIIEAADVTGQDPAEVAKAYFAVGSALDITWYISQISALPVENNWQALAREAFRDDVDWQQRAITIAVLQAGGGDSDVETRLALWMKQNDAMIERWRAMLVEIRAASGTDYAMYAVANRELNDVALSGQAVVPAAATAELELA

SEQ ID NO: 6 - DNA, 4887 bp, Pseudomonas fluorescens
  misc_feature: glutamate dehydrogenase enzyme 1.4.1.2 coding sequence
atggcgttcttcaccgcagccagcaaagccgacttccagcaccaactgcaagcggcactggcgcagcacatcagtgaacaggcactgccacaagtggcgctgttcgctgaacaattcttcggcatcatttccctggacgagctgacccaacgtcgcctctccgacctcgctggctgtactctctctgcgtggcgcctgcttgagcgcttcgatcacgcgcaaccgcaagtgcgcgtctacaaccccgattacgaacgtcacggctggcagtcgacccacaccgcggtcgaagtgctgcaccacgacttgccgttcctggtggactcggtgcgtaccgagctgaaccgtcgcggctacagcatccacaccctgcagaccaccgtgctgagcgtgcgtcgtggcagcaagggcgaattgctggaaatcctgccaaaaggcaccaccggcgaaggcgttctgcacgagtcgctgatgtacctggaaatcgaccgctgcgccaatgcggccgaattgaatgtgctgtccaaggaactggagcaggtcctgggtgaagtccgcgtcgcggtctccgatttcgagccgatgaaggccaaggtgcaggaaatcctcaccaagctcgataacagcgcattcgccgtcgatgccgacgaaaagaatgaaatcaagagcttcctggaatggctggtgggcaaccacttcaccttcctcggctacgaagagttcaccgttgtcgatcaggccgatggcggccacatcgaatacgaccagaactccttcctcggcctgaccaagatgctgcgcaccggtctgaccaacgaagaccgccacatcgaagactatgccgtgaagtacctgcgcgaaccgacactgctgtcgttcgccaaggcggcgcatccgagccgcgtgcaccgtccggcctacccggactacgtgtcgatccgcgaaatcgatgccgacggcaaagtgatcaaggaacaccgcttcatgggcctgtacacctcgtcggtgtatggcgaaagcgtgcgtgtcatcccgttcatccgccgcaaggtcgaggaaatcgagcgtcgctccggcttccaggccaaggctcacctgggcaaggaactggctcaggttctggaagtgctgccgcgtgacgatctgttccagaccccggtcgacgaactgttcagcaccgtgatgtcgatcgtgcagatccaggaacgcaacaagatccgcgtgttcctgcgtaaagacccgtacggtcgtttctgctactgcctggcctacgtgccgcgtgacatctactccaccgaagttcgccagaagatccagcaagtgctgatggagcgcctgaaagcctccgactgcgaattctggacgttcttctccgagtccgtgctggcccgcgtgcaactgatcttgcgcgtcgacccgaaaaaccgcatcgacatcgacccgctgcaactggaaaacgaagtgatccaggcctgccgcagctggcaggacgactacgctgccctgaccgttgaaaccttcggcgaagccaacggcaccaacgtgttggccgacttcccgaaaggcttcccggccggctaccgcgagcgtttcgcagcgcattcggccgtggtcgacatgcagcacttgctcaatctgagcgagaaaaagccgctggccatgagcttttaccagccgctggcctccggcccacgcgagctgcactgcaagctgtatcacgccgataccccgctggccctgtccgacgtgctgccgatcctggaaaacctcggcctgcgcgtgctgggtgagttcccgtaccgcctgcgtcataccaacggccgcgagttctggatccacgacttcgcgttcaccgctgccgaaggcctggacctggacatccagcaactcaacgacaccctgcaggacgcgttcgtccacatcgtccgtggcgatgccgaaaacgatgcgttcaaccgtctggtgctgaccgccggcctgccatggcgcgacgtggcgctgctgcgtgcctacgcccgctacctgaagcagatccgcctgggcttcgacctcggctacatcgccagcaccctgaacaaccacaccgacatcgctcgcgaactgacccggttgttcaagacccgtttctacctggcccgcaagctgggcagcgaggatctggacgacaagcaacagcgtctggaacaggccatcctgaccgcgctggacgacgttcaagtcctcaacgaagaccgcatcctgcgtcgttacctggacctgatcaaagcaaccctgcgcaccaacttctaccagaccgacgccaacggccagaacaagtcgtacttcagcttcaagttcaacccgcacttgattcctgaactgccgaaaccggtgccgaagttcgaaatcttcgtttactcgccacgcgtcgaaggcgtgcacctgcgcttcggcaacgttgctcgtggtggtctgcgctggtcggaccgtgaagaagacttccgtaccgaagtcctcggcctggtaaaagcccagcaagtgaagaactcggtcatcgtgccggtgggggcgaagggcggcttcctgccgcgtcgcctgccactgggcggcagccgtgacgagatcgcggccgagggcatcgcctgctaccgcatcttcatttcgggcctgttggacatcaccgacaacctgaaagacggcaaactggtaccgccggccaacgtcgtgcggcatgacgacgatgacccgtacctggtggtcgcggcggacaagggcactgcaaccttctccgacatcgccaacggcatcgccatcgactacggcttctggctgggtgacgcgttcgcgtccggtggttcggccggttacgaccacaagaaaatgggcatcaccgccaagggcgcgtgggtcggcgtacagcgccacttccgcgagcgcggcatcaatgtccaggaagacagcatcacggtggtcggcgtgggcgacatggccggtgacgtgttcggtaacggcctgttgatgtctgacaagctgcaactggttgctgcgttcaaccacctgcacatcttcatcgacccgaacccgaacccggccaccagcttcgccgagcgtcagcgcatgttcgatctgccgcgctcggcctggtccgactacgacaccagcatcatgtccgaaggcggcggcatcttctcgcgcagcgcgaagagcatcgccatctcgccacagatgaaagagcgcttcgacatccaggccgacaagctgaccccgaccgaactgctgaacgccttgctcaaggcgccggtggatctgctgtggaacggcggtatcggtacctacgtcaaagccagcaccgaaagtcacgccgatgtcggcgacaaggccaacgatgcgctgcgcgtgaacggcaacgaactgcgctgcaaagtggtgggcgagggcggtaacctcggcatgacccaattgggtcgtgtggagttcggtctcaatggcggcggttccaacaccgacttcatcgacaacgccggtggcgtggactgctccgaccacgaagtgaacatcaagatcctgctgaacgaagtggttcaggccggcgacatgaccgacaagcaacgcaaccagttgctggcgagcatgaccgacgaagtcggtggtctggtgctgggcaacaactacaagcagactcaggccctgtccctggcggcccgccgtgcttatgcgcggatcgccgagtacaagcgtctgatgagcgacctggagggccgtggcaagctggatcgcgccatcgagttcctgccgaccgaagagcaactggccgaacgcgttgccgaaggccatggcctgacccgtcctgagctgtcggtgctgatctcgtacagcaagatcgacctcaaggagcagctgctgggctccctggtgccggacgacgactacctgacccgcgacatggaaacggcgttcccgccgaccctggtcagcaagttctccgaagctatgcgtcgtcaccgcctcaagcgcgagatcgtcagcacccagatcgccaacgatctggtcaaccacatgggcatcaccttcgttcagcgactcaaagagtccacgggcatgaccccggcgaatgttgccggtgcgtatgtgattgttcgggatatcttccacctcccgcactggttccgtcagatcgaagcgctggactaccaggtttccgctgacgtgcagctggagctgatggacgagctgatgcgtctgggccgtcgcgctacgcgctggttcctgcgtgcccgtcgcaacgagcagaacgctgcccgtgacgtcgcgcatttcggtccgcacctcaaagagctgggcctgaagctggacgagctgctgagcggcgaaatccgcgaaaactggcaagagcgttatcaggcgtacgtcgccgccggtgttccggaactgctggcgcgtatggtggcggggacgacccacctctacacgctgctgccgatcatcgaagcggccgacgtgaccggccaggatccagccgaagtggccaaggcgtacttcgccgtgggcagcgcgctggacatcacctggtacatctcgcagatcagcgccttgccggttgaaaacaactggcaggccctggcccgtgaagcgttccgcgacgacgtcgactggcagcaacgcgcgattaccatcgccgttctgcaagcgggtggcggtgattcggacgtggaaacccgtctggcactgtggatgaagcagaacgacgccatgatcgaacgctggcgcgccatgctggtggaaatccgtgccgccagcggcaccgactacgccatgtacgcggtggccaaccgcgagctgaacgacgtggcgctgagcggtcaggcagttgtgcctgctgcggcgactgcggagcttgagcttgcttga

SEQ ID NO: 7 - protein, 387 aa, Pseudomonas savastanoi pv. Phaseolicola
  MISC_FEATURE: ethylene-forming enzyme (GenBank KPB44727.1)
MIHAPSRWGVFPSLGLCSPDVVWNEHPSLYMDKEETSMTNLQTFELPTEVTGCAADISLGRALIQAWQKDGIFQIKTDSEQDRKTQEAMAASKQFCKEPLTFKSSCVSDLTYSGYVASGEEVTAGKPDFPEIFTVCKDLSVGDQRVKAGWPCHGPVPWPNNTYQKSMKTFMEELGLAGERLLKLTALGFELPINTFTDLTRDGWHHMRVLRFPPQTSTLSRGIGAHTDYGLLVIAAQDDVGGLYIRPPVEGEKRNRNWLPGESSAGMFEHDEPWTFVTPTPGVWTVFPGDILQFMTGGQLLSTPHKVKLNTRERFACAYFHEPNFEASAYPLFEPSANERIHYGEHFTNMFMRCYPDRITTQRINKENRLAHLEDLKKYSDTRATGS

SEQ ID NO: 8 - DNA, 1161 bp, Pseudomonas savastanoi pv. Phaseolicola
  misc_feature: the sequence codes for ethylene-forming enzyme (GenBank KPB44727.1)
atgatacacgctccaagtagatggggagtatttccctcactagggttatgcagcccggacgttgtgtggaatgagcatccgagcctgtacatggacaaagaggaaaccagcatgaccaacctgcagacctttgaactgccgaccgaagtgaccggttgcgcggcggacatcagcctgggtcgtgcgctgattcaggcgtggcaaaaggatggtatcttccagattaaaaccgacagcgagcaggatcgtaagacccaagaagcgatggcggcgagcaagcaattttgcaaagagccgctgaccttcaaaagcagctgcgttagcgacctgacctacagcggttatgtggcgagcggcgaggaagttaccgcgggcaagccggatttcccggaaatttttaccgtgtgcaaggacctgagcgtgggcgatcagcgtgttaaagcgggttggccgtgccatggtccggttccgtggccgaacaacacctatcaaaagagcatgaaaacctttatggaggaactgggtctggcgggcgagcgtctgctgaaactgaccgcgctgggttttgaactgccgatcaacaccttcaccgacctgacccgtgatggctggcaccacatgcgtgtgctgcgtttcccgccgcagaccagcaccctgagccgtggtattggtgcgcacaccgactacggtctgctggtgattgcggcgcaagacgatgttggtggcctgtatatccgtccgccggtggagggcgaaaagcgtaaccgtaactggctgccgggcgagagcagcgcgggcatgtttgagcacgacgaaccgtggaccttcgttaccccgaccccgggtgtgtggaccgtttttccgggcgatattctgcagttcatgaccggtggccaactgctgagcaccccgcacaaggttaaactgaacacccgtgaacgtttcgcgtgcgcgtactttcacgagccgaacttcgaagcgagcgcgtatccgctgttcgagccgagcgcgaacgaacgtatccactacggcgagcacttcaccaacatgtttatgcgttgctatccggatcgtatcaccacccaacgtattaacaaagaaaaccgtctggcgcacctggaagacctgaagaaatacagcgacacccgtgcgaccggcagc

SEQ ID NO: 9 - protein, 808 aa, Artificial
  Synthetic amino acid sequence
MYKLVQTIVNSDEKNVLGDFILELGKDHKRYFLRNEILQAFADYCHQFPKPAYFYHSSSLGTFIQYTHEIILDGENTWFVVRPKIASQEVWLLSADLTKFELMTPKALLDVSDRLVKRYQPHILEIDLHPFYSAAPRIDDSRNIGQGLTVLNHYFCNQALTDPEYWIDALFQSLKRLEYNGIKLLISNHIHSGLQLTKQIKLALEFVSHLSPQTPYIKFKFHLQELGLEPGWGNNAARVRETLELLERLMDNPEPAILETFVSRICAVFRVVLISIHGWVAQEDVLGRDETLGQVIYVLEQARSLENKMRAEIKLAGLDTLGIKPHIIILTRLIPNCEGTFCNLPLEKVDGTENAWILRVPFAESRPEITNNWISKFEIWPYLEKFALDAEAELLKQFQGKPNLIIGNYSDGNLVAFILSRKMKVTQCNIAHSLEKPKYLFSNLYWQDLEAQYHFSAQFTADLISMNAADFIITSSYQEIVGTPDTMGQYESYKCFTMPNLYHVIDGIDLFSPKFNVVLPGVSENIFFPYNQTTNRESHRRQHIQDLIFHQEHPEILGKLDHPHKKPIFSVSPITSIKNLTGLVECFGKSEELQKHSNLILLTSKLHPDLGTNSEEIQEIAKIHAIIDQYHLHHKIRWLGMRLPLRDIAETYRVIADFQGIYIHFALYESFSRSILEAMISGLPTFTTQFGGSLEIIENHDQGFNLNPTDLAGTAKTIINFLEKCENYPEHWLENSQWMIERIRHKYNWNSHTNQLLLLTKMFSFWNFIYPEDNEARDRYMESLFHLLYKPIADHILSEHLSKIRNHN

SEQ ID NO: 10 - DNA, 2427 bp, Artificial
  Synthetic DNA sequence
atgtataaattagtgcaaactattgttaacagtgatgaaaaaaatgttttaggtgactttatcttagaattaggcaaggatcataaacgttactttttaagaaatgagattttacaagcttttgcagattattgtcaccaattcccaaaacccgcttatttttatcactcttcctctttagggacattcattcaatacacccatgaaataattttagatggtgaaaatacttggtttgtagttagaccaaagattgcgagtcaagaagtatggttattaagcgcggacttgactaagtttgagttaatgacaccgaaagcattattagatgtgagcgatcgcttagtaaagcgttatcaaccgcacattttagaaattgatctccatcccttttattcagcagcaccaagaattgatgattccagaaatattggccaaggtttaaccgttcttaatcattatttttgtaatcaagcattgacagatcctgaatattggattgacgcattatttcaatcattaaaaagattagaatataacggcatcaaattattaattagtaatcatattcattcaggtttgcaactaacaaagcaaatcaaactagcgttagaatttgtgagtcatttatccccccagacaccatatataaaatttaaatttcatcttcaagaactcggtttagaaccaggttggggtaataatgcagccagagtcagagaaaccttagaactgctggaaagactcatggataatcccgaacctgcaattttagaaacctttgtttctcgcatttgtgcagttttccgcgtcgtccttatttccatccatggttgggttgcacaagaagatgttttaggcagagatgaaacattaggacaagttatttatgttttagaacaagcccgcagtttagaaaataaaatgcgggcagaaattaaacttgcaggtttagatacattaggaattaaaccccatatcattatattaactcgactgattcccaattgtgaaggcacattttgtaacttaccattagaaaaagttgatggtacagaaaatgcttggattttgcgcgttccttttgcagaatctcgaccggaaattaccaacaactggatttctaaatttgaaatttggccttatttagaaaaatttgctcttgatgccgaagcagaacttttaaaacaattccaaggaaagcccaatctaattattggtaactacagtgacgggaacttagttgcttttattctctcccgaaaaatgaaagttacccaatgtaatattgcccattccctcgaaaaacctaaatatctatttagtaacttatattggcaagatttagaagcacaatatcacttttctgcccaatttaccgctgatttaatcagtatgaatgccgcagattttattatcacatcatcctatcaagaaattgtaggtacaccagatacaatgggacaatatgaatcttataaatgtttcaccatgcccaacttatatcatgtaattgatggcattgatttatttagccctaaattcaatgtggtattaccaggagtcagtgaaaatatattttttccctacaaccaaacaacaaatagagaatcccaccgtcgtcaacatatccaagacctaattttccatcaagaacacccagaaattctcggtaaattagatcatcctcataaaaaaccgatcttttccgttagtcccattacctcaattaaaaacctcacaggtttagttgaatgtttcggtaaaagtgaagaattacaaaaacatagtaacctaattttattaaccagtaaacttcatccagacttaggaacaaactccgaagaaattcaagaaatagcaaaaattcatgcgattattgatcaatatcatcttcaccataaaatccgctggttgggaatgcgtcttcctctccgcgatattgctgaaacctatcgtgtaattgccgattttcaagggatttatattcactttgccctttatgaatcctttagcagaagtattttagaagcaatgatttctggattaccaacttttacaactcaatttggtggttcattagaaattattgaaaaccatgatcaaggatttaacctcaaccccacagacttagcaggaacagccaaaacaattatcaacttcttagaaaaatgtgaaaattatccagaacattggctagaaaattctcaatggatgattgaacgcattcgccataaatataactggaattcccacacaaatcaactcctgttattaacgaaaatgtttagcttttggaacttcatctatcccgaagataacgaagccagagatcgttacatggaaagtttatttcatcttctttataaacctatagctgaccatattttatcagaacatctaagcaaaatcagaaatcataattaa

SEQ ID NO: 11 - protein, 709 aa, Artificial
  Synthetic amino acid sequence
MAAQNLYILHIQTHGLLRGQNLELGRDADTGGQTKYVLELAQAQAKSPQVQQVDIITRQITDPRVSVGYSQAIEPFAPKGRIVRLPFGPKRYLRKELLWPHLYTFADAILQYLAQQKRTPTWIQAHYADAGQVGSLLSRWLNVPLIFTGHSLGRIKLKKLLEQDWPLEEIEAQFNIQQRIDAEEMTLTHADWIVASTQQEVEEQYRVYDRYNPERKLVIPPGVDTDRFRFQPLGDRGVVLQQELSRFLRDPEKPQILCLCRPAPRKNVPALVRAFGEHPWLRKKANLVLVLGSRQDINQMDRGSRQVFQEIFHLVDRYDLYGSVAYPKQHQADDVPEFYRLAAHSGGVFVNPALTEPFGLTILEAGSCGVPVVATHDGGPQEILKHCDFGTLVDVSRPANIATALATLLSDRDLWQCYHRNGIEKVPAHYSWDQHVNTLFERMETVALPRRRAVSFVRSRKRLIDAKRLVVSDIDNTLLGDRQGLENLMTYLDQYRDHFAFGIATGRRLDSAQEVLKEWGVPSPNFWVTSVGSEIHYGTDAEPDISWEKHINRNWNPQRIRAVMAQLPFLELQPEEDQTPFKVSFFVRDRHETVLREVRQHLRRHRLRLKSIYSHQEFLDILPLAASKGDAIRHLSLRWRIPLENILVAGDSGNDEEMLKGHNLGVVVGNYSPELEPLRSYERVYFAEGHYANGILEALKHYRFFEAIA

SEQ ID NO: 12 - DNA, 2130 bp, Artificial
  Synthetic DNA sequence
gtggcagctcaaaatctctacattctgcacattcagacccatggtctgctgcgagggcagaacttggaactggggcgagatgccgacaccggcgggcagaccaagtacgtcttagaactggctcaagcccaagctaaatccccacaagtccaacaagtcgacatcatcacccgccaaatcaccgacccccgcgtcagtgttggttacagtcaggcgatcgaaccctttgcgcccaaaggtcggattgtccgtttgccttttggccccaaacgctacctccgtaaagagctgctttggccccatctctacacctttgcggatgcaattctccaatatctggctcagcaaaagcgcaccccgacttggattcaggcccactatgctgatgctggccaagtgggatcactgctgagtcgctggttgaatgtaccgctaattttcacagggcattctctggggcggatcaagctaaaaaagctgttggagcaagactggccgcttgaggaaattgaagcgcaattcaatattcaacagcgaattgatgcggaggagatgacgctcactcatgctgactggattgtcgccagcactcagcaggaagtggaggagcaataccgcgtttacgatcgctacaacccagagcgcaagcttgtcattccaccgggtgtcgataccgatcgcttcaggtttcagcccttgggcgatcgcggtgttgttctccaacaggaactgagccgctttctgcgcgacccagaaaaacctcaaattctctgcctctgtcgccccgcacctcgcaaaaatgtaccggcgctggtgcgagcctttggcgaacatccttggctgcgcaaaaaagccaaccttgtcttagtactgggcagccgccaagacatcaaccagatggatcgcggcagtcggcaggtgttccaagagattttccatctggtcgatcgctacgacctctacggcagcgtcgcctatcccaaacagcatcaggctgatgatgtgccggagttctatcgcctagcggctcattccggcggggtattcgtcaatccggcgctgaccgaaccttttggtttgacaattttggaggcaggaagctgcggcgtgccggtggtggcaacccatgatggcggcccccaggaaattctcaaacactgtgatttcggcactttagttgatgtcagccgacccgctaatatcgcgactgcactcgccaccctgctgagcgatcgcgatctttggcagtgctatcaccgcaatggcattgaaaaagttcccgcccattacagctgggatcaacatgtcaataccctgtttgagcgcatggaaacggtggctttgcctcgtcgtcgtgctgtcagtttcgtacggagtcgcaaacgcttgattgatgccaaacgccttgtcgttagtgacatcgacaacacactgttgggcgatcgtcaaggactcgagaatttaatgacctatctcgatcagtatcgcgatcattttgcctttggaattgccacggggcgtcgcctagactctgcccaagaagtcttgaaagagtggggcgttccttcgccaaacttctgggtgacttccgtcggcagcgagattcactatggcaccgatgctgaaccggatatcagctgggaaaagcatatcaatcgcaactggaatcctcagcgaattcgggcagtaatggcacaactaccctttcttgaactgcagccggaagaggatcaaacacccttcaaagtcagcttctttgtccgcgatcgccacgagactgtgctgcgagaagtacggcaacatcttcgccgccatcgcctgcggctgaagtcaatctattcccatcaggagtttcttgacattctgccgctagctgcctcgaaaggggatgcgattcgccacctctcactccgctggcggattcctcttgagaacattttggtggcaggcgattctggtaacgatgaggaaatgctcaagggccataatctcggcgttgtagttggcaattactcaccggaattggagccactgcgcagctacgagcgcgtctattttgctgagggccactatgctaatggcattctggaagccttaaaacactatcgcttttttgaggcgatcgcttaa

SEQ ID NO: 13 - DNA, 4186 bp, Artificial
  Synthetic promotor DNA sequence
  promoter (1)..(4186): LacIq promoter
caattgccctaagacagttgtcgtctttcgaagtctagttaacattaggggcgattctttgtttccactgagtggaagcaaacggtatcaaggttgcaggcagactcaaggtctagattgcttcacagcttgtgtggctatatttattatcttcattattgatggtagttgtgggtggatttaagatggaaaagtaacagataaatgtcgtctttaagggcgatctagatcgtatcgtttttaattcctaggtcggcatttattaatcaacctcgatacaatatttttttgtaaaaacttctagataaatgactcaagtctcattgaaagtctggggtgttgcctccccagtcaattcaagattaccaaggcctcgcatcgcctcttctattttgtttgaaggggacctaacgtgttgcgccaagctagttctcgacagagcatctcaagagcgcgttgctcgcggggggcaaacagttggagatcagccagctcttgcaagacttgttgggtgaggtggctggcaaagctaccggcatagcgcagtaagagactgtagtagcgaaattgcggttggccgttgcaatcgcggtgaaaggcagcaatttctgcttcgctgaggcagtagacaggggtattgaccggcacgatcgcgcgaggaatgccctgttcgccgtagcgatcgccgccctctgtttccgctaagctgccgatcaaaaccgtggagagcagcgtgcgggtttcattgattaaatcaggcgtgaatagtgggtcgggcccacttgaaagacgcggcccgttgttcagaaagaggggattaaacaactgcgagttgtagaccactccgatcgcccaatggcgatcgccttccaaacgaacaaagctgccaaatccatagctctcggcggctggtggattggagacatccatgtcgtcatccacttggacaacgtagtcacagtgcgagttggatttgacaactttgccgaggcgcatggtgctgccagtgttacaaccaattaaccaattctgacatatggacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtttcactggtgaaaagaaaaaccaccctggcgcccaatacgcaaaccgcctctccccgcgcgttggccgattcattaatgcagctggcacgacaggtttcccgactggaaagcgggcagtgagctttgttgacaattaatcatccggctcgtataatgtgtggaattgtgagcggataacaagaaggagatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaattgttcagaacgctcggtcttgcacaccgggcgttttttctttgtgagtccaggtaccaatcaatctcccccaagtcaagcggcgctgagacccagtgtctgccggtgagtcagtcttggcaagcaaactgtgcctttgcgatttcttaccctacgcagctccgggatcgatcggaggtaaccaaggctacggacaatggcgcgggcaccagctgttggtaaactgaggagcgatcgccgcttcagtccaaaggctatgacgcaaaaatccgttgttattgctccgtccattctgtcagcggatttcagccgcttgggcgacgatgtccgcgctgttgaccaggctggcgctgactggattcacgtcgatgtgatggatggtcgcttcgtccctaacatcaccattggaccgctgatcgttgaagcgctgcgcccggtgacccaaaagccgttggacgtccacttgatgatcgtcgagccggaaaaatatgtgccggatttcgcgaaagcaggggctgacatcatctcggtccaagcagaagcttgcccccacctgcaccgcaacttggctcagatcaaagacctcggcaagcaagcaggcgtcgtcctcaacccctctaccccagtcgaaaccctggaatacgtgctggagttgtgcgacctgattttgatcatgagcgtcaaccctggcttcggtggtcagagtttcatcccagctgtcctgccgaaaatccgtaagctgcgcgccatgtgcgatgagcgtggccttgatccttggattgaagtcgatggcggcttgaaagccaataacacttggcaggtgctggaagccggtgctaacgcaattgtggcgggctcggcagtcttcaacgcgccggactatgctgaagcgatcgcggcgattcgcaacagcaagcgtcctgaacttgtcactgcttaggcttctcgctcaacgctcagtggagcaatctgaatcttgcagcccttcagtggatcagtctgctgaggggttttgctttaggatgggcgatcgcgagtagggacacggatcgctggta

(19) United States
(12) Patent Application Publication
(10) Pub. No.: US 2023/0167466 A1
(43) Pub. Date: Jun. 1, 2023

(54) METHOD FOR PRODUCING ETHYLENE FROM CARBON DIOXIDE

(71) Applicant: Oxy Low Carbon Ventures, LLC, Houston, TX (US)

(72) Inventors: Donald H. POWERS, Houston, TX (US); Tahereh KARIMI, Houston, TX (US); Robert L ZELLER, III, Seabrook, TX (US); John PACE, Cypress, TX (US); Michael A. GARMON, Manvel, TX (US); Truong Huu NGUYEN, Houston, TX (US)

(21) Appl. No.: 17/912,903

(22) PCT Filed: Mar. 19, 2021
(86) PCT No.: PCT/US2021/023163

§ 371 Date: Sep. 20, 2022

Related U.S. Application Data
(60) Provisional application No. 63/052,664, filed on Jul. 16, 2020.
(60) Provisional application No. 62/992,689, filed on Mar. 20, 2020.

(51) Int. Cl. C12P 5/02; C12M 1/107; C12M 1/00; C12N 9/04; C12N 9/06; C12N 9/10

(52) U.S. Cl. CPC C12P 5/026; C12M 21/04; C12M 23/58; C12M 47/18; C12N 9/0006; C12Y 101/01042; C12N 9/0016; C12Y 104/01002; C12N 9/1066

(57) ABSTRACT

A process comprising (i) providing a gaseous stream including greater than 1% by volume carbon dioxide; (ii) providing water; (iii) converting the carbon dioxide and the water to an organic intermediate and oxygen gas in the presence of light; (iv) separating the oxygen gas from the organic intermediate; and (v) converting the organic intermediate to ethylene and carbon dioxide after said step of separating the oxygen gas from the organic intermediate.

This application claims the benefit of U.S. Provisional Application Ser. No. 62/992,689 filed on Mar. 20, 2020, and U.S. Provisional Application Ser. No. 63/052,664 filed on Jul. 16, 2020.

### FIELD OF THE INVENTION

Embodiments of the present invention provide a method and system for converting carbon dioxide to ethylene.

### BACKGROUND OF THE INVENTION

Concerns over the long-term availability of fossil fuels coupled with concerns over atmospheric carbon dioxide levels has intensified the development of biosynthetic methods for producing petroleum-based products via carbon dioxide fixation. That is, photosynthetic processes are used to convert carbon dioxide to useful organic compounds that can be used as fuels or organic building blocks for larger organic molecules.

For example, U.S. Pat. No. 7,807,427 teaches the use of photosynthetic organisms, such as genetically-modified cyanobacteria, to convert carbon dioxide into intermediate products such as glucose and acetic acid. In a subsequent step, methanogenic bacteria are used to convert the intermediate products to methane. The methane can be collected and stored for use as fuel.

Ethylene is widely known as the most significant chemical feedstock for many industries, and several methodologies have been proposed to biosynthesize ethylene through carbon dioxide fixation.

It is known that Pseudomonas syringae synthesizes ethylene using the TCA cycle intermediate alpha-ketoglutarate in a single-step reaction catalyzed by an ethylene-forming enzyme, which is referred to as “efe.” With the goal of directly producing ethylene from carbon dioxide, U.S. Pat. No. 9,309,541 discloses methods for expressing and overexpressing efe genes in hosts such as Synechocystis to generate strains that are capable of producing ethylene phototrophically. In other words, through bioengineering, photoautotrophs are modified to convert the products of carbon fixation to ethylene in a single step reaction. While current research has focused on modifying photoautotrophs to produce ethylene, harvesting the ethylene can present challenges. As outlined in Eckert et al., Ethylene forming enzyme and bioethylene production, BIOTECHNOLOGY FOR BIOFUELS 2014, 7:33, when O₂ is co-produced with ethylene in a photosynthetic system, there is an important safety issue regarding the flammability of the ethylene in the presence of the O₂, which requires engineering designs to mitigate the risk.

### SUMMARY OF THE INVENTION

One or more embodiments of the present invention provide a process comprising (i) providing a gaseous stream including greater than 1% by volume carbon dioxide; (ii) providing water; (iii) converting the carbon dioxide and the water to an organic intermediate and oxygen gas in the presence of light; (iv) separating the oxygen gas from the organic intermediate; and (v) converting the organic intermediate to ethylene and carbon dioxide after said step of separating the oxygen gas from the organic intermediate.

Yet other embodiments of the present invention provide a system for producing ethylene, the system comprising (i) a first bioreactor including photosynthetic microorganisms that convert carbon dioxide to an organic intermediate, said first bioreactor having a carbon dioxide inlet and an outlet for the organic intermediate; and (ii) a second bioreactor in fluid communication with the first bioreactor and including microorganisms that convert the organic intermediate produced in the first bioreactor to ethylene, said second bioreactor having an outlet for gaseous materials including ethylene, and said second bioreactor having an outlet for fluid materials including unreacted organic intermediate.

### BRIEF DESCRIPTION OF THE DRAWINGS

FIG. 1 is a schematic view of a system for practicing embodiments of the invention.

FIG. 2 is a schematic view of a subsystem for delivering carbon dioxide within embodiments of the present invention.

FIG. 3 is a schematic view of an alternate system including a second photosynthetic bioreactor for practicing embodiments of the invention.

FIG. 4 is a schematic view of a system for including a oxygen-fuel combustion system for practicing embodiments of the invention.

FIG. 5 is a schematic view a system including upstream carbon dioxide purification for practicing embodiments of the invention.

FIG. 6 is a schematic view of an ethylene-purification and compression scheme applicable to one or more embodiments of the invention.

FIG. 7 is a schematic view of an alternate system including carbon dioxide membrane separation for practicing one or more embodiments of the present invention.

FIG. 8 is a schematic view of an alternate system including a single bioreactor for practicing embodiments of the present invention.

### DETAILED DESCRIPTION OF ILLUSTRATIVE EMBODIMENTS

Embodiments of the invention are based, at least in part, on the discovery of a method for biosynthesizing ethylene from carbon dioxide at industrially significant levels. According to the embodiments of the invention, carbon dioxide is first photosynthetically converted to an organic intermediate with the by-product production of oxygen gas. The by-product oxygen gas is then separated from the organic intermediate, and the organic intermediate is then biologically converted to ethylene in the appreciable absence of the by-product oxygen gas. While current technology associated with biosynthesizing ethylene is focused on single-step synthesis, the two-step method of the present invention addresses safety concerns associated with the co-production of ethylene and oxygen gas at industrially significant levels. Also, the bioconversion of the organic intermediate to the ethylene produces by-product carbon dioxide, which impacts overall carbon efficiency. Moreover, the amount of carbon dioxide in the ethylene product stream is significant relative to most carbon dioxide input streams (e.g. flue gas), and therefore the carbon dioxide within the ethylene product stream can be a valuable resource if appropriately managed. Accordingly, embodiments of the invention provide management solutions for the by-product carbon dioxide including, but not limited to, photosynthetically converting the carbon dioxide to organic intermediates, which can be recycled back to the step of biologically converting the organic intermediate to ethylene. Still further, the step of biologically converting organic intermediate to ethylene can be efficiently achieved on a commercial scale by using industrial reactors such as continuously-stirred tank reactors, which operate at or near steady-state, which dictates incomplete consumption of the organic intermediate, loss of carbon efficiency, and the loss of valuable raw materials. Embodiments of the invention therefore provide solutions to these problems by appropriately managing the effluent streams from the reactor in which organic intermediate is converted to ethylene.

### Process and System Overview

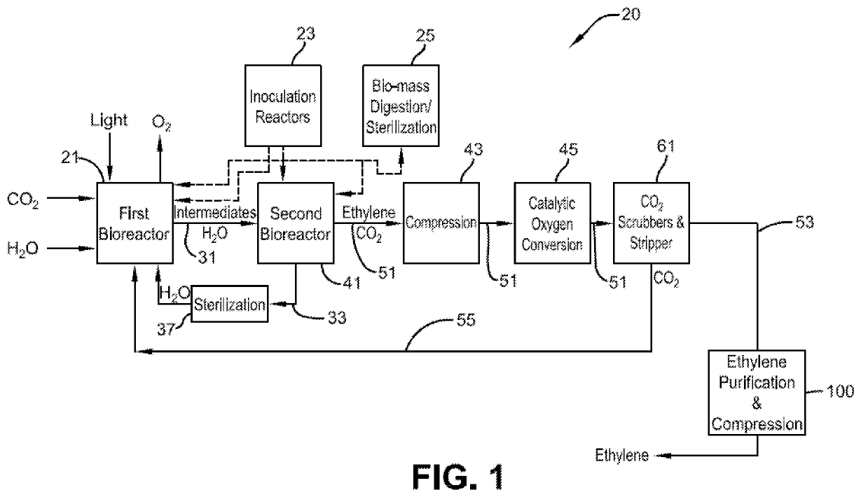

Embodiments of the invention can be described with reference to FIG. 1, which depicts a system 20 for converting carbon dioxide to ethylene. The system includes a first bioreactor 21 followed in series by a second bioreactor 41. First bioreactor 21 is in fluid communication, either directly or indirectly, with second bioreactor 41 via intermediate-product conduit 31. Second bioreactor 41 is also in fluid communication with first bioreactor 21 via intermediate-recycle conduit 33. A carbon dioxide separator 61 is downstream of second bioreactor 41 and is in in fluid communication, either directly or indirectly, with second bioreactor 41 via conduit 51. Carbon dioxide separator 61 may also be in fluid communication with first bioreactor 21, either directly or indirectly, via carbon dioxide-recycle conduit 53.

According to embodiments of the invention, first bioreactor 21 includes a photosynthetic organism culture (i.e. photosynthetic microorganisms) that converts carbon dioxide and water fed to bioreactor 21 to an organic intermediate. This conversion takes place in the presence of light energy that is supplied to first bioreactor 21. The synthesis of the organic intermediate takes place in the presence of excess water, which acts as reaction medium, and the excess water acts as the carrier for the intermediate product stream. In one or more embodiments, the organic intermediate is soluble in the water. As the skilled person will appreciate, the photosynthetic organism culture can be supplied to bioreactor 21 from an inoculation reactor 23.

Oxygen gas is produced as a by-product during formation of the organic intermediate within bioreactor 21. According to aspects of the present invention, the oxygen gas is separated from the organic intermediate prior introducing the intermediate product stream to second bioreactor 41. For example, the oxygen gas can be vented out of first bioreactor 21 together with other volatiles within the reactor such as nitrogen gas.

The organic intermediate is transferred from first reactor 21, either directly or indirectly, within an intermediate-product stream to second bioreactor 41 via intermediate-product conduit 31. In one or more embodiments, the intermediate-product stream can be filtered as the stream exits bioreactor 21. During operation, filtering of the intermediate-product stream as the stream leaves bioreactor 21 can prevent transfer of any media that is used to immobilize the photosynthetic microorganisms and thereby help prevent transfer of the microorganisms from first bioreactor 21 to second bioreactor 41.

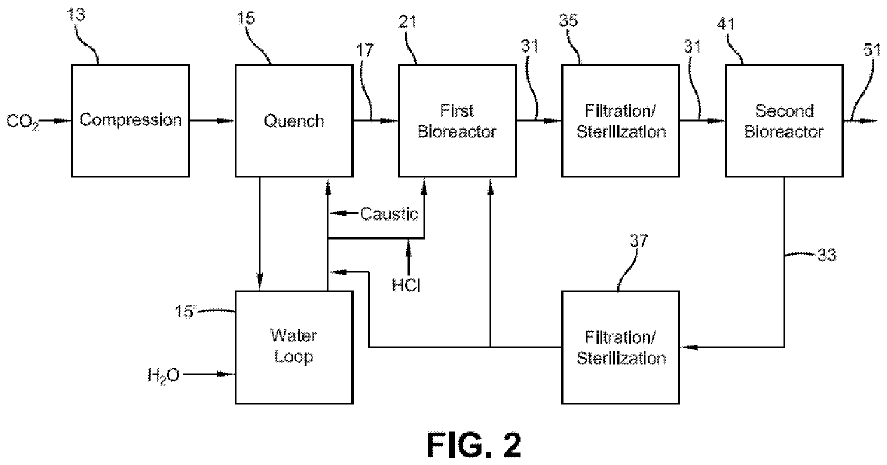

In addition to or in lieu of filtering the intermediate product stream as the stream leaves bioreactor 21, the intermediate-product stream can be filtered and/or sterilized at one or more intermediate units positioned between bioreactor 21 and bioreactor 41. For example, and with reference to FIG. 2, optional sterilization unit 35 can be positioned between first bioreactor 21 and second bioreactor 41. Unit 35 may include a filtration unit. In addition to or in lieu of a filtration unit, unit 35 may include a centrifugation unit. Or, in other embodiments, unit 35 may include, in addition to or in lieu of filtration or centrifugation, a clarification unit (e.g. a settling tank). In lieu of or in addition to filtration, centrifugation, and/or clarification, unit 35 may include a sterilization unit. For example, the sterilization unit may take advantage of UV sterilization, heat, or gamma radiation to treat the intermediate-product stream, which may be done in order to prevent the introduction of any live microorganisms from first bioreactor 21 into second bioreactor 41.

In one or more embodiments, second bioreactor 41 includes an ethylene-producing organism culture (i.e. ethylene-producing organisms) that converts the organic intermediate to ethylene.

As the skilled person appreciates, several sub-systems can be designed for introducing the microorganism cultures to the respective bioreactors. Those skilled in the art can readily design appropriate systems for accomplishing these goals. For example, and with reference to Figures, the appropriate microorganisms can be supplied to bioreactor 21 and/or bioreactor 41 from an inoculation unit 23, which may also be referred to as inoculation reactor 23. Inoculation unit 23 may include separate chambers or vessels for the respective microorganisms, or separate units may be provided for the respective microorganisms. Likewise, it may be desirable to remove biomass from one or more of the bioreactors. In one or more embodiments, the systems of the present invention may include a biomass digestion unit 25 where biomass obtained from either or both of the bioreactors can be removed from any immobilization support media and removed from the system. During operation, bio-mass digestion unit 25 can be in fluid communication with either or both of bioreactors 21, 41, or the bio-mass (optionally together with the immobilization materials) can be manually removed from the respective reactors. In one or more embodiments, the biomass can be converted to nutrients, such as amino acids, and returned to the bioreactors as a source of nutrient for the microorganisms. Alternatively, the biomass can be removed from the system and directed toward other uses such as fertilizers and the like.

Carbon dioxide is produced as a by-product of ethylene synthesis within bioreactor 41, and the ethylene and carbon dioxide are removed from second bioreactor 41 as a gaseous product stream. A liquid effluent stream also exits second bioreactor 41. This liquid effluent stream may include water and unreacted organic intermediates, and the stream can be routed back to first bioreactor 21 via organic intermediate-recycle conduit 33. As best shown in FIG. 2, the liquid effluent stream including water and unreacted organic intermediates can undergo filtration and/or sterilization at filtration/sterilization unit 37. This filtration and/or sterilization may take advantage of the same types of technologies as unit 35 and therefore the discussion above with respect to unit 35 is incorporated herein. As the skilled person appreciates, it may be useful to ensure that ethylene-producing microorganisms do not migrate into first bioreactor 21.

The gaseous product stream exiting second bioreactor 41 is routed downstream of second bioreactor 41 to carbon dioxide separator 61, which may also be referred to as carbon dioxide separation unit 61, either directly or indirectly, via conduit 51. Within separator 61, which may also be referred to as separator system 61, carbon dioxide is separated from the gaseous product stream to provide a concentrated ethylene stream, which may also be referred to as an ethylene-rich stream, which is carried by conduit 53. This ethylene-rich stream can be routed to downstream purification and pressurization units 100, which will be discussed in greater detail herein. Separator 61 also produces a concentrated carbon dioxide stream, which may also be referred to as a purified carbon dioxide stream, and this concentrated carbon dioxide stream can be routed back to first bioreactor 21 via conduit 55.

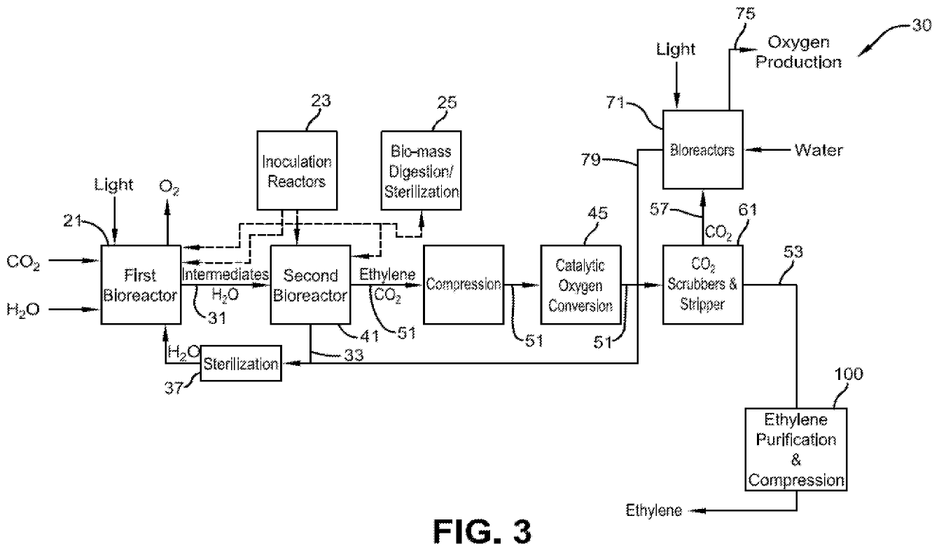

In alternative embodiments, which may be described with reference to FIG. 3, the purified carbon dioxide stream produced by carbon dioxide separator 61 can be routed, via conduit 57, to an intermediary bioreactor 71 (which may be referred to as second photosynthetic bioreactor 71) that includes a photosynthetic organism culture that photosynthetically converts the carbon dioxide an organic intermediate and oxygen gas. Advantageously, since the carbon dioxide feed stream to bioreactor 71 is a purified carbon dioxide stream (via carbon dioxide separator 61), the gaseous by-product stream leaving second photosynthetic bioreactor 71 includes a relatively pure oxygen gas stream, which can be routed via conduit 75. The skilled person will appreciate, within the context of this invention, that relatively pure oxygen streams include those streams that are substantially devoid of nitrogen gas and argon gas, which would otherwise require complicated and expensive processes to separate (e.g. air separation techniques) the nitrogen and argon from the oxygen gas. The presence of carbon dioxide, however, within the relatively pure oxygen gas streams defined herein is not deleterious and therefore may be present within the relatively pure oxygen steams, unless otherwise stated, since carbon dioxide can be more readily separated from the oxygen gas streams. Consistent with first bioreactor 21, intermediary bioreactor 71 produces an effluent stream, which may include organic intermediate and water, that can be routed back to first bioreactor 21 and/or second bioreactor 41 via conduit 79. As generally shown, this effluent stream may undergo filtration and/or sterilization at unit 37 as described with reference to FIG. 2.

As shown throughout the drawings, the gaseous stream exiting second bioreactor 41, which is routed via conduit 51, can optionally undergo one or more treatments or manipulations prior to carbon dioxide separation at unit 61. For example, the stream can be pressurized at compression unit 43. In addition to or in lieu of pressurization, the gaseous stream can optionally undergo treatment to remove oxygen at oxygen-removal unit 45. Since carbon dioxide can be generated at oxygen removal unit 45, it may be beneficial to position oxygen-removal unit 45 upstream of carbon dioxide separation unit 61 where the carbon dioxide produced at unit 45 can be removed.

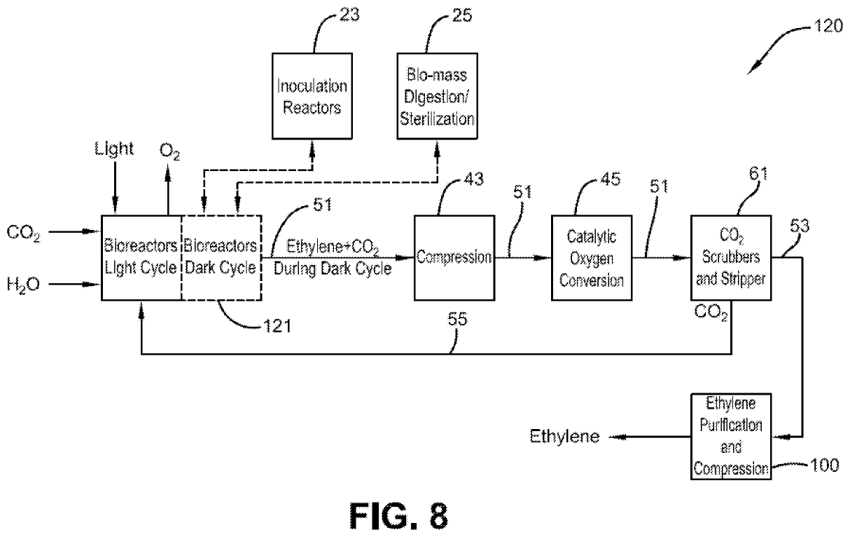

Yet another embodiment of the present invention can be described with reference to FIG. 8. As shown, process 120 includes a single vessel 121, which may also be referred to as integrated bioreactor 121, in lieu of the two bioreactors 21, 41 shown with respect to the other systems described above. During operation, light energy supplied to bioreactor 121 is controlled in a manner to produce a light cycle and a dark cycle. During operation, photosynthetic microorganisms within integrated bioreactor 121 convert carbon dioxide to an organic intermediate during the light cycle, and then during the dark cycle, ethylene-forming microorganisms within integrated bioreactor 121 convert the organic intermediate to ethylene during the dark cycle. Oxygen gas can be removed from bioreactor 121 during its production during the light cycle, and ethylene can be removed from bioreactor 121 during its production during the dark cycle. Consistent with other embodiments, ethylene may be coproduced with carbon dioxide, and the ethylene and carbon dioxide can be separated in downstream processes as described above (e.g. at carbon dioxide scrubber 61).

Turning back to FIG. 2, the processes of the present invention may include conditioning of the carbon dioxide input stream (i.e. conditioning of the stream prior to providing the stream to bioreactor 21). In one or more embodiments, the carbon dioxide input stream, which is carried by conduit 11, may be pressurized at compressor 13. In one or more embodiments, pressurization of the carbon dioxide input stream (e.g. within compressor 13) achieves sufficient pressure to overcome counter forces within first bioreactor 21 so that inert gases (e.g. nitrogen) within the input stream can ultimately enter the head space of the reactor. In one or more embodiments, the carbon dioxide input stream is pressurized to a pressure of from about 2 to about 20 psig, in other embodiments from about 3 to about 18 psig, and in other embodiments from about 5 to about 15 psig.

Also, as best shown in FIG. 2, the carbon dioxide input stream can be cooled at quencher 15 prior to delivery to bioreactor 21 via conduit 17. As the skilled person will appreciate, quencher 15 may include a water-cooled unit that includes a quench water loop 15′, which may include one or more heat exchangers for cooling the water. In one or more embodiments, the carbon dioxide input stream is cooled to a temperature below that which would otherwise have a deleterious impact on the microorganism culture within bioreactor 21. In one or more embodiments, carbon dioxide input stream is cooled to a temperature of from about 10 to about 80° C., in other embodiments from about 20 to about 60° C., and in other embodiments from about 30 to about 50° C. prior to delivery to bioreactor 21.

Inasmuch as the carbon dioxide input streams of one or more embodiments may include appreciable amounts of water, and at least a portion of the water will be condensed via the cooling cycle at quencher 15, water from quencher 15 can be fed to first bioreactor 21, which consumes substantial amounts of water. In one or more embodiments, the water employed at quencher 15 and/or the water stream routed to first bioreactor 21 can be treated with caustic to adjust the pH of the water. In one or more embodiments, the water employed at quencher 15 and/or water routed to first bioreactor 21 from quencher 15 is adjusted to a pH of greater than 5.5, in other embodiments greater than 6.0, and in other embodiments greater than 6.5 (e.g. in the range 5.5-8.0 or 6.0-7.5). It is anticipated that caustic treatment of the water will form carbonates, such as sodium carbonate, which not only benefits first bioreactor 21 relative to pH control, but also offers a source of additional carbon dioxide in the form of sodium carbonate and/or sodium bicarbonate. Those skilled in the art can readily adjust the conditions and/or provide additional ingredients (e.g. hydrochloric acid) to yield a desirable balance between sodium carbonate and sodium bicarbonate. In particular embodiments, the caustic soda provided to the quench water within quencher 15 derives from other processes that can be integrated with practice of the present invention. For example, ethylene purification can use caustic soda and/or generate sodium carbonate that can be integrated with quencher 15.

In yet other embodiments, the liquid effluent stream exiting second bioreactor 41, which as described relative to other embodiments can be routed back to first bioreactor 21, can optionally be routed to quencher 15. In one or more embodiments, liquid effluent stream exiting second bioreactor 41 is first treated at sterilization station 37 prior to routing to quencher 15.

### Carbon Dioxide Feed Stream to First Bioreactor

The process of the present invention can advantageously convert carbon dioxide from a variety of gaseous sources, which may be referred to as carbon dioxide input streams, to useful intermediates that can be converted to ethylene. In one or more embodiments, the carbon dioxide is provided to the system by a carbon dioxide input stream that includes greater than 1% vol, in other embodiments greater than 3% vol, in other embodiments greater than 5% vol, and in other embodiments greater than 10% vol carbon dioxide. In one or more embodiments, the carbon dioxide input stream is or derives from an exhaust stream of a combustion process (i.e. a flue gas stream). As the skilled person appreciates, the composition of the exhaust stream can vary based upon several factors including the design of the combustion process and fuel being burned in the combustion process. For example, the flue gas stream can derive from coal-fired furnaces, gas-fired furnaces, turbine-powered generators, and oxy-fuel combustion processes.

In one or more embodiments, the carbon dioxide input stream can derive from an exhaust stream of an oxy-fuel combustion process, which may also be referred to as oxycombustion. Those skilled in the art appreciate that these processes include the combustion of fuels (e.g. hydrocarbons) in the substantial absence of nitrogen and argon. For example, these processes may include combustion processes where substantially pure oxygen (i.e. substantially free of nitrogen and argon gas), or a mixture of pure oxygen and recycled flue gas, is fed to the combustion process. As a result, the combustion products are mostly carbon dioxide and water and substantially low in nitrogen by-products or argon. Advantageously, because the carbon dioxide input stream from an oxycombustion process includes significant levels of carbon dioxide, and is substantially devoid of nitrogen and oxygen, the gaseous by-product stream from photosynthetic bioreactor would include substantially high concentrations of oxygen gas together with any unreacted carbon dioxide. The gaseous by-product stream from photosynthetic bioreactor can then be recycled back to the oxycombustion unit as fuel within the oxycombustion process, with any unreacted carbon dioxide providing cooling to the oxycombustion process.

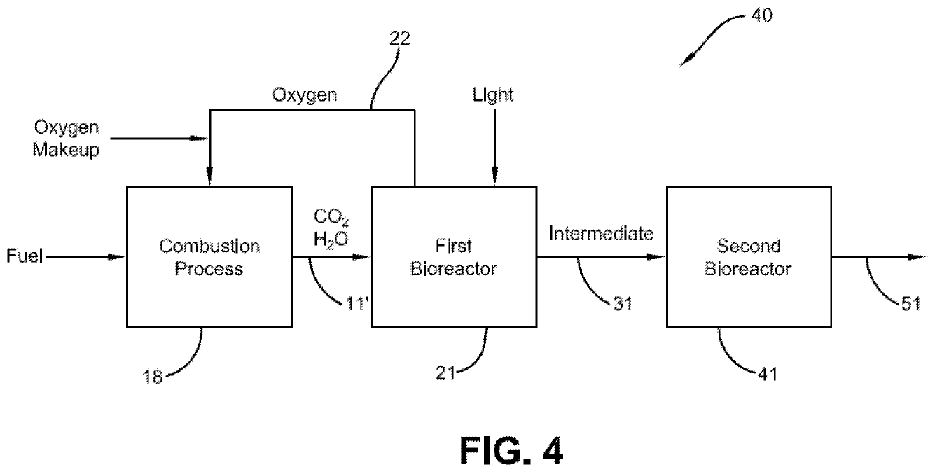

For example, and with reference to FIG. 4, an embodiment of the invention includes a carbon dioxide input stream from an oxycombustion unit 18. As with the previous embodiments, carbon dioxide is photosynthetically converted to an organic intermediate with by-product oxygen gas with first bioreactor 21. The by-product oxygen gas stream, which includes oxygen gas and unreacted carbon dioxide, is routed, via conduit 22, to oxycombustion unit 18. Also, although not shown, the carbon dioxide input stream from oxycombustion unit 18 can be cooled and pressurized as described above with respect to the other embodiments (see e.g. FIG. 2). The intermediate products produced in first bioreactor 21 are routed downstream to second bioreactor 41 in a manner consistent with other embodiments.

In yet other embodiments, relatively pure carbon dioxide streams can be fed to first bioreactor 21. Relatively pure carbon dioxide streams can be obtained from several sources and generally include those streams that contain greater than 90 vol %, in other embodiments greater than 95 vol %, and in other embodiments greater than 99 vol % carbon dioxide. As explained above, where relatively pure carbon dioxide streams are used, the process of the present invention produces relatively pure oxygen gas streams (i.e. substantially free of nitrogen or argon gas) as a by-product output from first bioreactor 21. These relatively pure oxygen gas streams can be used, for example, in industrial applications such as the oxychlorination of ethylene.

In one or more embodiments, a relatively pure carbon dioxide stream is produced as a step of the present invention and used as an input stream. For example, an input stream containing carbon dioxide can be purified and/or concentrated prior to introducing the stream to first bioreactor 21. In one or more embodiments, the carbon dioxide input stream can be purified by using, for example, amine scrubbing and stripping techniques. As with one or more of the previous embodiments, by providing a purified carbon dioxide stream to the first bioreactor, relatively high-grade oxygen gas streams can be produced as the by-product stream exiting the first bioreactor. The skilled person will appreciate that a variety of carbon dioxide removal and separation techniques can be used in addition to or in lieu of amine scrubbing and stripping techniques to purify and/or concentrate a carbon dioxide stream. These techniques include, but are not limited to, membrane separation, solid sorbents, and the use of other solvent chemistries such as potassium carbonate.

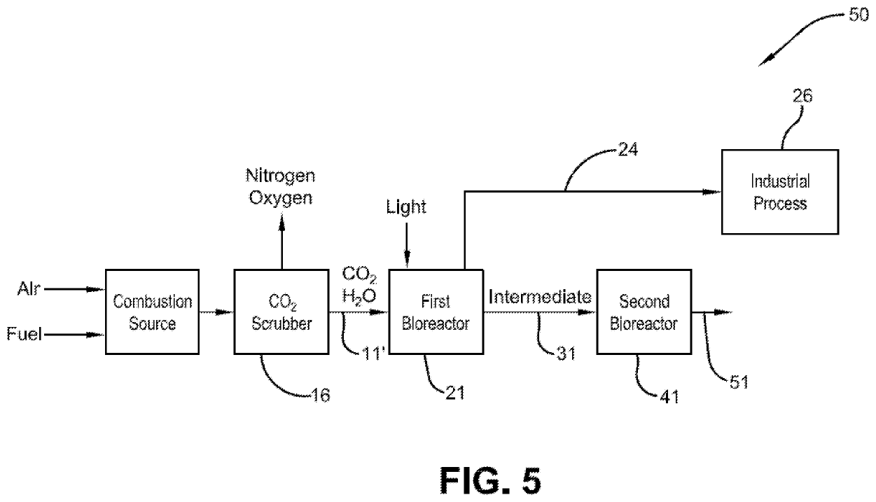

An exemplary embodiment can be described with reference to FIG. 5, which shows a system 50 including first bioreactor 21 that receives a carbon dioxide input stream from a carbon dioxide purification unit 16. Purification unit 16 produces a purified carbon dioxide stream that is introduced to first bioreactor 21 via conduit 11′. Although not shown, the carbon dioxide input stream from the combustion unit can be cooled and pressurized as described above with respect to the other embodiments (see e.g. FIG. 2). The carbon dioxide is photosynthetically converted to an organic intermediate with by-product oxygen gas in bioreactor 21. The by-product oxygen gas is routed, either directly or indirectly, via conduit 24, to, for example, an industrial process 26 requiring relatively high purity oxygen. In one or more embodiments, ethylene produced at bioreactor 41 can also be routed to industrial process 26. Although not shown in FIG. 5, it will be appreciated that the ethylene streams from second bioreactor 41 undergo downstream processing as described with regard to other embodiments including, but not limited to, oxygen gas conversion, carbon dioxide removal (and recycle back to the photosynthetic bioreactor), and ethylene purification.

Again, in those embodiments where the photosynthetic bioreactor yields a relatively pure oxygen by-product stream, which can derive from the use of relatively pure carbon dioxide streams, the relatively pure oxygen streams can be used in industrial applications. In one or more embodiments, the gaseous stream exiting the photosynthetic bioreactor can undergo carbon dioxide removal to remove any unreacted carbon dioxide in the oxygen gas stream. Then, the oxygen gas stream can be routed to the desired industrial applications. For example, the oxygen gas stream from these embodiments can be routed to an oxychlorination unit, where ethylene is reacted with hydrochloric acid in the presence oxygen gas. Within this example, the ethylene can derive from the ethylene-producing bioreactor. It will be appreciated that the ethylene stream from the ethylene-producing bioreactor will undergo carbon dioxide removal and ethylene purification as described with regard to the other embodiments.

### Carbon Dioxide Separation

In one or more embodiments, downstream carbon dioxide separation (e.g. at carbon dioxide separation unit 61) may be conducted by using conventional amine scrubbing/stripping. A variety of other carbon dioxide separation techniques can used including, but not limited to, solvent separation using potassium carbonate, membrane separation, and solid sorbent separation.

As the skilled person appreciates, amine scrubbing and stripping techniques or methods generally include absorption (i.e. scrubbing) of carbon dioxide by organic amines within a water carrier, and the subsequent regeneration or release of the carbon dioxide from the organic amine (i.e. stripping). These systems and the techniques for their use are well known in the art as described in U.S. Publ. Nos. 2009/0038314, 2009/0156696, and 2013/0244312, which are incorporated herein by reference. Reference can also be made to the Engineering Data Book, Vol. II, Sections 17-26; Gas Processors Suppliers Assoc. (1994).

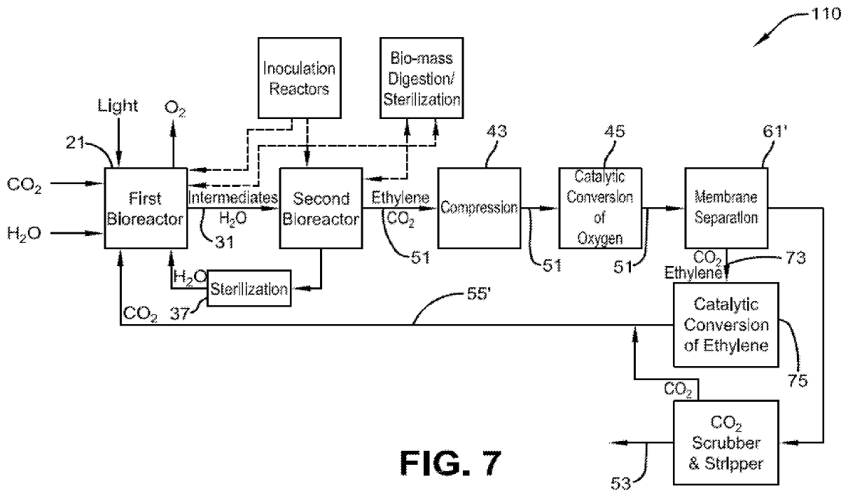

In lieu of or in addition to amine scrubbing/stripping, membrane separation techniques may be employed. As the skilled person appreciates, these membranes may include polymer or inorganic microporous membranes that allow the passage of carbon dioxide through the permeant. These membranes and the techniques for their use are well known as described in U.S. Publ. Nos. 2008/0173179 and 2013/0312604, which are incorporated herein by reference. In practicing the present invention, where membrane separation is used in lieu of amine scrubbing/stripping techniques, it may be desirable to removal residual ethylene that may migrate into the permeant stream and therefore would otherwise be routed back to the fixation reactor (i.e. the first bioreactor) and then ultimately migrate into the oxygen streams. In this regard, reference is made to FIG. 7, which shows alternate system 20′ including membrane separation unit 61′ with the permeant stream of carbon dioxide routed downstream via conduit 73 to ethylene treatment unit 75, which may be adapted to catalytically treat the stream to remove (e.g. via catalytic combustion) any residual ethylene within the permeant stream prior to the permeant stream being routed back to bioreactor 21 via conduit 51′.

### Ethylene Purification

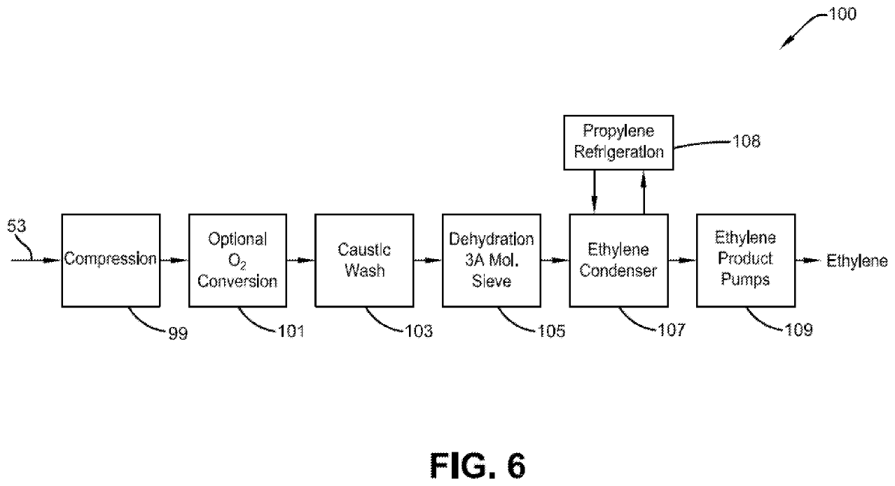

As described above, carbon dioxide separation (e.g. within separator 61) of the ethylene-containing product stream of second bioreactor 41 produces an ethylene-rich stream that is carried by conduit 53. This ethylene-rich stream can undergo purification and optional compression for later use and optional transport within subprocess 100, which is best described with reference to FIG. 6. In one or more embodiments, subprocess 100 treats ethylene-rich steam, which is provided via conduit 53, using one or more techniques. For example, the ethylene-rich stream may undergo oxygen gas removal at optional oxygen gas removal unit 101. Within this unit, residual oxygen gas within the ethylene-rich stream is consumed by, for example, catalytic combustion of a portion of the ethylene. In one or more embodiments, subprocess 100 may include polishing of the ethylene stream at caustic washing unit 103, which removes any residual carbon dioxide (e.g. reduce the level of carbon dioxide to less than 10 ppm, or less 5 ppm, or less than 3 ppm). Downstream of caustic washing unit 103, the ethylene-rich stream may undergo dewatering at dewatering unit 105, which may include a dehydration unit using molecular sieves. Following dewatering, the ethylene-rich stream can be condensed at condenser 107, which can be cooled by propylene refrigeration unit 108. The skilled person will appreciate that the sequence of the various purification steps can be altered depending on a number of factors. Also, the skilled person will appreciate that the various steps of the purification process may be conducted with a goal of achieving a desired pressure, which may be required for use or transport (e.g. via a pipeline). To this end, the ethylene-rich stream may be pressurized before, after, or in between two more purification steps. For example, as shown in FIG. 6, the stream may be pressurized at compression unit 99. Likewise, further pressurization can take place at ethylene product pumps 109.

### Photosynthetic Microorganisms

As set forth above, the first bioreactor contains a photosynthetic organism culture containing one or more types of photosynthetic microorganisms that converts the carbon dioxide and water into an organic intermediate in the presence of light energy. In various embodiments, the photosynthetic microorganisms may be naturally occurring. In other embodiments, the photosynthetic microorganisms may be genetically modified for improved production of a desired organic intermediate. In one or more embodiments, the photosynthetic microorganisms utilized with the first bioreactor may include photosynthetic bacteria such as cyanobacteria. As those skilled in the art appreciate, photosynthetic bacteria consume carbon dioxide and water in the presence of light to fix carbon. Advantageously, the main products of the metabolic pathway of cyanobacteria during aerobic conditions are oxygen and organic intermediates, such as sugars. One of ordinary skill in the art will be able to select a suitable photosynthetic microorganism, without undue experimentation, to produce a desired organic intermediate.

In one or more embodiments, the desired organic intermediate includes sucrose, dextrose, xylose, glucose, fructose, alpha-ketoglutarate, or a mixture thereof.

Exemplary photosynthetic microorganisms include, without limitation, cyanobacteria, algae, and purple bacteria. Useful types of cyanobacteria include photosynthetic prokaryotes that carry out oxygenic photosynthesis. Cyanobacteria useful for the purposes outlined herein are generally well known in the art. (See, e.g., Donald Bryant, The Molecular Biology of Cyanobacteria, published by Kluwer Academic Publishers (1994), the disclosure of which in incorporated herein by reference in its entirety). Representative examples include cyanobacteria in the genus Synechococcus such as Synechococcus lividus and Synechococcus elongatus; and cyanobacteria in the genus Synechocystis such as Synechocystis minervae and Synchocystis Sp PCC 6803. In this regard, U.S. Pat. No. 7,807,427 is incorporated herein by reference in its entirety. Examples of synthetic microorganisms that can be used include those disclosed in U.S. Pat. No. 10,196,627, which is incorporated herein by reference in its entirety. Still other examples include those microorganisms disclosed in U.S. Pat. Nos. 9,914,947 and 9,309,541, which are incorporated herein by reference in their entirety.

In one or more embodiments, the cyanobacteria are genetically modified to express one or more foreign genes encoding one or more enzymes that provide for enhanced production of target organic intermediates. In one or more embodiments, the target organic intermediates include sucrose, dextrose, xylose, glucose, fructose, and alpha-ketoglutarate. As will be apparent to those of skill in the art, the particular genes added to the genome of the cyanobacteria (and enzymes produced) will depend upon the particular target organic intermediate.

In one or more embodiments, the modified photosynthetic microorganism includes a modified nucleotide sequence that produces an enzyme that forms alpha-ketoglutarate from carbon dioxide. In certain embodiments, this modified photosynthetic microorganism expresses an alpha-ketoglutarate permease protein (AKGP) by expressing a non-native AKGP forming nucleotide sequence. In one or more embodiments, the modified microorganism produces a greater amount of the enzyme than produced by a control microorganism lacking the modified nucleotide sequence. This amount may be greater than 1%, in other embodiments greater than 50%, and in other embodiments greater than 75% of the amount produced by a control microorganism lacking the modified nucleotide sequence.

In one or more embodiments, alpha-ketoglutarate (aKG) can be produced by oxidative decarboxylation of isocitrate by isocitrate dehydrogenase (ICD), or by oxidative deamination of glutamate by glutamate dehydrogenase (GDH). Target enzymes for cloning and aKG production in Cyanobacteria may include ICD Enzyme: 1.1.1.42, coding sequence of P. Fluorescens ICD (SEQ ID NO: 1, SEQ ID NO: 2), ICD Enzyme: 1.1.1.42, coding sequence of Synechococcus elongatus PCC794 (SEQ ID. NO: 3, SEQ ID NO: 4), and GDH Enzyme: 1.4.1.2, coding sequence of P. Fluorescens (SEQ ID NO: 5, SEQ ID NO: 6).

In one or more embodiments, the enzyme for forming alpha-ketoglutarate is selected from isocitrate dehydrogenase (ICD) protein, a glutamate dehydrogenase (GDH) protein, or a combination thereof.

In certain embodiments, the modified photosynthetic microorganism expresses an ICD protein having an amino acid sequence at least 98%, or at least 95%, or at least 90%, or at least 85% identical SEQ ID NO: 1 by expressing a modified ICD protein nucleotide sequence having a nucleotide sequence at least 98%, or at least 95%, or at least 90%, or at least 85% identical to SEQ ID NO: 2. In certain embodiments, the modified microorganism expresses an ICD protein having an amino acid sequence at least 98%, or at least 95%, or at least 90%, or at least 85% identical SEQ ID NO: 3 by expressing a modified ICD protein nucleotide sequence having a nucleotide sequence at least 98%, or at least 95%, or at least 90%, or at least 85% identical to SEQ ID NO: 4. In certain embodiments, the modified microorganism expresses a GDH protein having an amino acid sequence at least 98%, or at least 95%, or at least 90%, or at least 85% identical SEQ ID NO: 5 by expressing a modified GDH protein nucleotide sequence having a nucleotide sequence at least 98%, or at least 95%, or at least 90%, or at least 85% identical to SEQ ID NO: 6.

In one or more embodiments, the organic intermediate is sucrose. In some of these embodiments, for example, the Cyanobacteria (Synechococcus elongatus, Synechocystis) can be engineered to produce sucrose to serve as a substrate for the growth of the ethylene producing microorganisms. Various methods for the engineering of Synechococcus elongatus PCC 7942 to produce sucrose can include activation of one gene (cscB) and deletion of one gene (GlgC).

In one or more of these embodiments, the modified photosynthetic microorganism expresses a sucrose synthase protein. In one or more embodiments, the sucrose synthase protein has an amino acid sequence at least 98%, or at least 95%, or at least 90%, or at least 85% identical to SEQ ID NO: 9 by expressing a modified sucrose synthase protein nucleotide sequence having a nucleotide sequence at least 98%, or at least 95%, or at least 90%, or at least 85% identical to SEQ ID NO: 10. In one or more of these embodiments, the modified microorganism expresses a sucrose phosphate synthase protein. In one or more embodiments, the sucrose phosphate synthase protein has an amino acid sequence at least 98%, or at least 95%, or at least 90%, or at least 85% identical to SEQ ID NO: 11 by expressing a modified sucrose phosphate synthase protein nucleotide sequence having a nucleotide sequence at least 98%, or at least 95%, or at least 90%, or at least 85% identical to SEQ ID NO: 12.

As used herein, sequence identity is the relationship between two or more amino acid (polypeptide or protein) sequences or two or more nucleic acid (polynucleotide) sequences, as determined by comparing the sequences. Sequence identities or similarities are typically compared over the whole length of the respective sequences. The skilled person appreciates that “identity” refers to the degree of sequence relatedness between amino acid or nucleic acid sequences, as the case may be, as determined by the match between strings of such sequences. “Similarity” between two amino acid sequences is determined by comparing the amino acid sequence and its conserved amino acid substitutes of one polypeptide to the sequence of a second polypeptide. The skilled person can readily calculate “identity” and “similarity” by various known methods. For example, methods to determine identity and similarity are codified in publicly available computer programs, such as the BestFit, BLASTP (Protein Basic Local Alignment Search Tool), BLASTN (Nucleotide Basic Local Alignment Search Tool), FASTA (Altschul, S. F. et al., J. Mol. Biol. 215:403-410 (1990), publicly available from NCBI and other sources (BLAST® Manual, Altschul, S., et al., NCBI NLM NIH Bethesda, Md. 20894), and EMBOSS (European Molecular Biology Open Software Suite). Exemplary parameters for amino acid sequences comparison using EMBOSS are gap open 10.0, gap extend 0.5, Blosum matrix. Exemplary parameters for nucleic acid sequences comparison using EMBOSS are gap open 10.0, gap extend 0.5, DNA full matrix (DNA identity matrix). As the skilled person understands, the DNA/protein sequences among different species can be compared to determine the homology of sequences using online data such as Gene bank, KEG, BLAST and Ensemble. The skilled person may also take into account so-called “conservative” amino acid substitutions, which refers to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Substitutional variants of the amino acid sequence disclosed herein are those in which at least one residue in the disclosed sequences has been removed and a different residue inserted in its place. Preferably, the amino acid change is conservative. Preferred conservative substitutions for each of the naturally occurring amino acids are as follows: Ala to ser; Arg to lys; Asn to gln or his; Asp to glu; Cys to ser or ala; Gln to asn; Glu to asp; Gly to pro; His to asn or gln; Ile to leu or val; Leu to ile or val; Lys to arg; gln or glu; Met to leu or ile; Phe to met, leu or tyr; Ser to thr; Thr to ser; Trp to tyr; Tyr to trp or phe; and, Val to ile or leu.

Unless otherwise noted, the term “adapted” or “codon adapted” refers to “codon optimization” of polynucleotides as disclosed herein, the sequence of which may be native or non-native, or may be adapted for expression in other microorganisms. Codon optimization adapts the codon usage for an encoded polypeptide towards the codon bias of the organism in which the polypeptide is to be expressed. Codon optimization generally helps to increase the production level of the encoded polypeptide in the host cell.

In certain embodiments, the modified photosynthetic microorganism includes a delta-glgc (Δglgc) mutant microorganism lacking expression of a glucose-1-phosphate adenylyltransferase protein. Similarly, cyanobacterial cells that lack a functional ADP-glucose pyrophosphorylase enzyme are known as described in U.S. Pat. Nos. 9,309,541 and 9,309,541, which are incorporated herein by reference in their entirety.

### Ethylene-Producing Microorganisms

As described above, the ethylene-producing organisms include those organisms that naturally produce or are genetically modified to produce ethylene by consuming the organic intermediate produced in the photosynthetic bioreactor. In one or more embodiments, microorganisms utilized with the second bioreactor include those microorganisms that express, or have been genetically modified to express, an ethylene-forming enzyme (efe) gene. These microorganisms may be referred to herein as efe-forming microorganisms. Microorganisms that produce, or have been modified to produce, ethylene are well known in the art and any microorganism capable of producing ethylene from the target organic intermediate under the reaction conditions described may be used. In one or more embodiments, the desired microorganisms do not produce anything else that would otherwise interfere with the methods described herein.

Exemplary efe-forming microorganisms include Pseudomonas syringae, Pseudomonas syringae pv. Glycinia, and Penicillium digitatum, which all naturally express an efe. In other embodiments, the microorganisms within the second reactor include modified microorganisms that express or overexpress an efe gene such as that naturally found in Pseudomonas syringae or Penicillium digitatum. In these embodiments, one or more copies of one or more efe genes are transfected into a host microorganism using any one of numerous methods known in the art for doing so. Useful hosts microorganisms may include, without limitation, Escherichia coli (E. Coli), Saccharomyces cerevisiae, Pseudomonas putida, Trichoderma viride, and Trichoderma reesei.

In one or more embodiments, the modified microorganism for producing efe may be produced as set forth in Wang, J. P., et al., “Metabolic engineering for ethylene production by inserting the ethylene-forming enzyme gene (efe) at the 16S rDNA sites of Pseudomonas putida KT2440” Biosource Technology, (2010) 101: 6404-6409, the disclosure of which is incorporated herein by reference in its entirety. In these embodiments, efe gene is cloned from P. syringae pv. glycinea ICMP2189 and inserted into one or more 16S rDNA sites of a Pseudomonas pudita KT2440 host using double crossover recombination.

As set forth above, in one or more embodiments, the microorganism that produces the efe enzyme will include genetically engineered microorganisms. In one or more embodiments, the efe-forming microorganism is a modified microorganism that includes within its DNA one or more foreign nucleotide sequence that produces efe when expressed. In one or more embodiments, the modified microorganism produces a greater amount of the efe enzyme than produced by a control microorganism lacking the modified nucleotide sequence. This amount may be greater than 5%, in other embodiments greater than 50%, and in other embodiments greater than 75% of the amount produced by a control microorganism lacking the modified nucleotide sequence. In various embodiments, the genetically modified microorganisms will be modified to contain two or more copies of the foreign nucleotide sequence that produces efe when expressed, further improving ethylene production.

In one or more embodiments, the polynucleotide coding for the Pseudomonas savastanoi pv. Phaseolicola efe protein (GenBank: KPB44727.1, SEQ ID NO: 8) can be cloned into a pET-30a(+) vector plasmid. The corresponding nucleotide sequences can also be codon adapted for expression in E. coli (SEQ ID NO: 7) and to contain an optional His tag at the C-terminal end followed by a stop codon and HindIII site. An NdeI site can also be used for cloning at the 5-prime end, where the NdeI site contains an ATG start codon. In various embodiments, E. coli BL21(DE3) competent cells can be transformed with the recombinant plasmid.

In some embodiments, ann Ampicillin cassette can be activated by an IPTG inducible promoter (pTrc) in the presence of a Lad gene; the Lad gene can be regulated by a LacIq promoter (SEQ ID NO: 13).

In certain embodiments, the ethylene-forming recombinant microorganism expresses an efe protein having an amino acid sequence at least 95%, or at least 90%, or at least 80% identical to SEQ ID NO: 7 by expressing a non-native efe protein nucleotide sequence having a nucleotide sequence at least 95%, or at least 90%, or at least 80% identical to SEQ ID NO: 8.

The skilled person appreciates that it may be beneficial to immobilize the microorganisms in order to increase yields and assist in the management of the microbes within the process (e.g. assist in separating the microorganisms from the product streams or reaction media). In one or more embodiments, the microorganisms are immobilized to a support media such as, but not limited to, a high surface area support media. Useful high surface area support materials may include sponges, fibrous material, bio-balls, ceramic filters, and the like.

### First Bioreactor (Photosynthetic Bioreactor)

With reference again to the Figures, first bioreactor 21 may include a single reaction vessel or it may include a plurality (i.e. two or more) reaction vessels that may operate in a complementary fashion. For example, the two or more reactor vessels may operate in parallel or in series to facilitate the desired photosynthetic reaction.

The skilled person generally appreciates the appropriate conditions that should be maintained with first bioreactor 21 to sustain the microorganisms and promote the desired photosynthetic reaction. In one or more embodiments, water is both a reactant and serves as the reaction medium within first bioreactor 21.

In one or more embodiments, the reactor medium within the photosynthetic bioreactor is maintained at a temperature of from about 25 to about 70° C., in other embodiments from about 35 to about 60° C., and in other embodiments from about 40 to about 50° C. In these or other embodiments, the reaction medium within the photosynthetic bioreactor is maintained at a pH of from about 5.0 to about 8.5, in other embodiments from about 5.5 to about 8.0, and in other embodiments from about 6.0 to about 7.0.

In one or more embodiments, the photosynthetic bioreactor is substantially devoid of microorganisms that produce or are adapted to produce an efe gene.

In one or more embodiments, the first bioreactor includes at least one inlet for the introduction of at least reactant (e.g. carbon dioxide) into the bioreactor. In these or other embodiments, the first bioreactor includes at least one outlet for removing at least one product or at least one by-product from the bioreactor. In one or more embodiments, first bioreactor 21 includes an outlet for gaseous product/by-product and an outlet for liquid effluent. In one or more embodiments, bioreactor 21 is a closed system but for the inlets and outlets. In other embodiments, bioreactor 21 is an open system. In one or more embodiments, the first bioreactor is selected from a continuous stirred tank reactor, a gas lift reactor, a loop reactor, and fluidized bed reactor. In one or more embodiments, the first bioreactor has a capacity of greater than 10,000 gallons, in other embodiments greater than 100,000 gallons, in other embodiments greater than 1,000,000 gallons.

### Second Bioreactor (Ethylene-Producing Bioreactor)

With reference again to the Figures, second bioreactor 41 may include a single reaction vessel or it may include a plurality (i.e. two or more) reaction vessels that may operate in a complementary fashion. For example, the two or more reactor vessels may operate in parallel or in series to facilitate the desired reaction of converting the intermediate to ethylene.

The skilled person generally appreciates the appropriate conditions that should be maintained with the second bioreactor to sustain the microorganisms and promote the desired ethylene-forming reaction. In one or more embodiments, water serves as the reaction medium in the second reactor.

In one or more embodiments, the reactor medium within the ethylene-forming bioreactor is maintained at a temperature of from about 25 to about 70° C., in other embodiments from about 35 to about 60° C., and in other embodiments from about 40 to about 50° C. In these or other embodiments, the reaction medium within the ethylene forming bioreactor is maintained at a pH of from about 6.0 to about 9.5, in other embodiments from about 6.5 to about 9.0, and in other embodiments from about 7.0 to about 8.0.

In one or more embodiments, the ethylene forming bioreactor is substantially devoid of microorganisms that produce or are adapted to produce oxygen. For example, the second bioreactor is devoid or substantially devoid of photosynthetic microorganisms (e.g. microorganisms that operate by the Calvin Cycle).

In one or more embodiments, the ethylene-forming bioreactor is maintained under anaerobic conditions. In one or more embodiments, the ethylene-forming bioreactor is maintained in the substantial absence of light energy.

In one or more embodiments, the second bioreactor includes at least one inlet for the introduction of the organic intermediate product stream into the bioreactor. In these or other embodiments, the second bioreactor includes at least one outlet for removing the product (e.g. gaseous outlet for ethylene gas) and at least one by-product from the second bioreactor (e.g. carbon dioxide). In one or more embodiments, the second bioreactor also includes an effluent outlet for the removal of liquid effluent (e.g. water and unreacted organic intermediate). In one or more embodiments, the second bioreactor is selected from a continuous stirred tank reactor, a loop reactor, and fluidized bed reactor. In one or more embodiments, the second bioreactor has a capacity of greater than 10,000 gallons, in other embodiments greater than 100,000 gallons, in other embodiments greater than 1,000,000 gallons. In one or more embodiments, the second bioreactor is adapted to provide a closed system but for the reactant inlet and product or by-product outlet.

In one or more embodiments, the concentration of microorganisms within the second reactor may be quantified based upon the dry cell weight per unit volume of the reactor. For example, in one or more embodiments, the microorganism concentration in the second reactor is greater than 10, in other embodiments greater than 50, and in other embodiments greater than 100 grams dry cell weight per liter.

### Techniques for Forming Recombinant Microorganisms

In certain embodiments, the nucleotide sequence for expressing the intermediate forming enzyme or for the efe is inserted into a microbial expression vector. In various embodiments, the microbial expression vector may include a bacterial vector plasmid, a nucleotide guide of a homologous recombination system, an antibiotic-resistant system, an aid system for protein purification and detection, a CRISPR CAS system, a phage display system, or a combination thereof.

As set forth above, in one or more embodiments, multiple copies of the efe expressing nucleotide sequence may be inserted into the ethylene forming microorganisms. Similarly, multiple copies of the intermediate enzyme expressing nucleotide sequence may be inserted into the photosynthetic microorganisms. The number of copies of a gene inserted into a vector and/or a host genome is referred to herein as the “copy number.” In certain embodiments, the efe expressing nucleotide sequence has a copy number in the microbial expression vector of greater than 1, in other embodiments greater than 10, in other embodiments greater than 100, and in other embodiments greater than 250. As will be apparent, expressing multiple copies of the efe expressing nucleotide sequence can increase ethylene yields, thus reducing the volume and cost of ethylene production on a commercial scale.

In certain embodiments, the microbial expression vector includes at least one microbial expression promoter. As will be understood by those of skill in the art, the microbial expression promotor is a nucleotide sequence that initiates transcription of a later, usually adjacent, sequence of DNA and may be constitutive or inducible. In certain embodiments, the at least one microbial expression promoter may include, without limitation, a light sensitive promoter, a chemical sensitive promoter, a temperature sensitive promoter, a Lac promoter, a T7 promoter, a CspA promoter, a lambda PL promoter, a lambda CL promoter, a continuously producing promoter, a psbA promoter, or a combination thereof. In certain embodiments, at least one promoter inducer may be added to the bioreactors or the reaction media within the bioreactors to control the amount of the organic intermediate and/or the amount of the ethylene being produced. In certain embodiments, the promoter inducer includes lactose, xylose, IPTG, cold shock, heat shock, or a combination thereof.

In one or more embodiments, the ICD and GDH genes may be synthesized using gBlocks™ gene fragments cloned into a pSyn6 plasmid construct (pSyn6_ICD and pSyn6_GDH). For cloning into a pSyn6 plasmid, the S. elongatus ICD coding sequence is flanked by an N-terminal HindIII and C-terminal BamHI recognition sites (SEQ ID NO: 4). Using the plasmid constructs, the ICD and GDH genes can be cloned into an unmodified S. elongatus or an S. elongatus Δglgc mutant strain (see Example 2). Between 1-3 copies of the target genes may be transformed. Cloning of the ICD and GCH genes can be confirmed by PCR and sequencing. aKG synthesis and quantification can be evaluated by SDS-PAGE, Western Blot, and Ethylene production assays.

In one or more embodiments, creating glycogen mutant strains of Cyanobacteria will change the bacteria's pathway to produce, and also secrete, higher concentrations of keto acids such as aKG.Glycogen mutant Cyanobacteria can be generated by creating glycogen deficient strains via mutations of the glgc gene (Δglgc). For example, an Ampicillin Resistance (AmpR) gene can be synthesized using gBlocks™ and incorporated into a plasmid construct. The plasmid construct can be transformed into a wild type Cyanobacteria (e.g., Synechocystis, Synechococcus elongatus 2973, Synechococcus elongatus 2434). A portion of the wild type glgc gene can then be replaced by the AmpR gene to create the mutant strains. The Δglgc mutant strains can be confirmed by growth in AmpR containing media, followed by PCR and sequencing.

### Maintaining Level of Volatile Gases

In one or more embodiments, the second reactor includes safe levels of oxygen gas. In particular, the head space in the second reactor and the gaseous outlet stream of the second reactor include safe levels of oxygen gas relative to ethylene. As the skilled person appreciates, commercially acceptable levels of oxygen gas within ethylene streams can be defined by the lower explosion limit (LEL), which takes into account the level of ethylene present. In one or more embodiments, the amount of oxygen within the second reactor (i.e. within the head space of the reactor or in the gaseous outlet stream) is less than the acceptable LEL, in other embodiments less than 80% of the acceptable LEL, and in other embodiments less than 50% of the acceptable LEL.

### Process Characteristics

As described herein, the process of the invention is effective for converting carbon dioxide to ethylene at high carbon efficiency while addressing safety concerns associated with the co-production of ethylene and oxygen by using biosynthetic processes.

In one or more embodiments, the process of the present invention produces ethylene at a production rate of greater than 100, in other embodiments greater than 500, in other embodiments greater than 1000, in other embodiments greater than 1500, in other embodiments greater than 2000, and in other embodiments greater than 2500 μmol/gCDW/hour, where CDW refers to cell dry weight.

Various modifications and alterations that do not depart from the scope and spirit of this invention will become apparent to those skilled in the art. This invention is not to be duly limited to the illustrative embodiments set forth herein.

APPENDIX

SEQ ID NO: 1-
MGYKKIQVPAVGDKITVNADHSLNVPDNPIIPFIEGDGIGVDVSPVMIKVVDAAVEKAYGGKRKISWMEVYAGEKATQ
VYDQDTWLPQETLDAVKDYVVSIKGPLTTPVGGGIRSLNVALRQQLDLYVCLRPVVWFEGVPSPVKKPGDVDMVIFRE
NSEDIYAGIEWKAGSPEATKVIKFLKEEMGVTKIRFDQDCGIGIKPVSKEGTKRLVRKALQYVVDNDRKSLTIVHKGNIM
KFTEGAFKDWGYEVAKEEFGAELLDGGPWMKFKNPKTGREVVVKDAIADAMLQQILLRPAEYDVIATLNLNGDYLSD
ALAAEVGGIGIAPGANLSDTVAMFEATHGTAPKYAGKDQVNPGSVILSAEMMLRHLGWTEAADLIIKGTNGAIKAKTV
TYDFERLMEGATLVSSSGFGEALIKHM
SEQ ID NO: 2-
ATGGGTTACAAGAAGATTCAGGTTCCAGCCGTCGGCGACAAAATCACCGTCAACGCAGACCATTCTCTCAATGTCC
CTGATAACCCGATCATTCCCTTCATCGAAGGTGACGGCATTGGCGTCGACGTCAGCCCTGTGATGATCAAAGTGGT
TGATGCTGCCGTAGAGAAAGCCTACGGGGGTAAGCGCAAGATTTCCTGGATGGAGGTTTATGCTGGCGAAAAAG
CAACTCAGGTCTATGACCAGGACACCTGGCTGCCCCAGGAAACCCTGGACGCGGTCAAGGATTACGTGGTCTCCA
TCAAAGGCCCGCTGACCACTCCGGTCGGTGGCGGCATCCGTTCCCTCAACGTCGCCCTGCGCCAACAGCTCGATCT
CTATGTCTGCCTTCGCCCTGTGGTGTGGTTCGAAGGTGTGCCGAGCCCGGTGAAAAAGCCTGGCGACGTCGACAT
GGTGATCTTCCGCGAGAACTCCGAAGACATTTATGCCGGTATCGAATGGAAAGCCGGCTCCCCTGAGGCCACCAA
GGTCATCAAATTCCTGAAAGAAGAAATGGGCGTCACCAAGATCCGTTTCGACCAGGATTGCGGCATCGGCATCAA
GCCGGTTTCCAAAGAAGGCACCAAGCGTCTGGTGCGCAAGGCGCTGCAATACGTGGTGGACAACGACCGCAAGT
CGCTGACCATCGTGCACAAGGGCAACATCATGAAATTCACCGAAGGTGCCTTCAAGGACTGGGGCTACGAGGTG
GCGAAGGAAGAATTCGGCGCCGAGCTGCTCGATGGCGGCCCATGGATGAAATTCAAGAACCCGAAAACCGGCCG
CGAAGTCGTCGTCAAGGACGCCATCGCCGACGCCATGCTCCAGCAGATCCTGCTGCGTCCGGCCGAATACGATGT
GATCGCCACCCTCAACCTCAACGGTGACTACCTGTCCGACGCCCTGGCGGCGGAAGTGGGCGGTATCGGTATCGC
GCCGGGTGCCAACCTGTCCGACACCGTAGCCATGTTCGAGGCGACCCACGGTACTGCGCCGAAATATGCCGGCAA
GGACCAGGTCAACCCGGGTTCGGTGATTTTGTCGGCGGAAATGATGCTGCGCCACCTGGGCTGGACCGAGGCGG
CCGACCTGATCATCAAGGGCACCAATGGCGCCATCAAGGCCAAGACCGTGACCTACGACTTCGAACGTCTGATGG
AAGGCGCCACACTGGTGTCTTCTTCGGGCTTCGGTGAAGCGCTGATCAAGCACATGTAA
SEQ ID NO: 3-
MYEKIQPPSEGSKIRFEAGKPIVPDNPIIPFIRGDGTGVDIWPATERVLDAAVAKAYGGQRKITWFKVYAGDEACDLYGT
YQYLPEDTLTAIREYGVAIKGPLTTPIGGGIRSLNVALRQIFDLYACVRPCRYYTGTPSPHRTPEQLDVVVYRENTEDIYLGI
EWKQGDPTGDRLIKLLNEDFIPNSPSLGKKQIRLDSGIGIKPISKTGSQRLIRRAIEHALRLEGRKRHVTLVHKGNIMKFTE
GAFRDWGYELATTEFRTDCVTERESWILANQESKPDLSLEDNARLIEPGYDAMTPEKQAAVVAEVKAVLDSIGATHGN
GQWKSKVLVDDRIADSIFQQIQTRPGEYSVLATMNLNGDYISDAAAAVVGGLGMAPGANIGDEAAIFEATHGTAPKH
AGLDRINPGSVILSGVMMLEYLGWQEAADLITKGISQAIANREVTYDLARLMEPAVDQPLKCSEFAEAIVKHFDD
SEQ ID NO: 4-
AGGGCAAGCTTATGTACGAGAAGATTCAACCCCCTAGCGAAGGCAGCAAAATTCGCTTTGAAGCCGGCAAGCCGA
TCGTTCCCGACAACCCGATCATTCCCTTCATTCGTGGTGACGGCACTGGCGTTGATATCTGGCCCGCAACTGAGCG
CGTTCTCGATGCCGCTGTCGCTAAAGCCTATGGCGGTCAGCGCAAAATCACTTGGTTCAAAGTCTACGCGGGTGAT
GAAGCCTGCGACCTCTACGGCACCTACCAATATCTGCCTGAAGATACGCTGACAGCGATCCGCGAGTACGGCGTG
GCAATCAAAGGCCCGCTGACGACGCCGATCGGTGGTGGCATTCGATCGCTGAACGTGGCGCTACGGCAAATCTTC
GATCTCTATGCCTGCGTCCGCCCCTGTCGCTACTACACCGGCACACCCTCGCCCCACCGCACGCCCGAACAACTCGA
TGTGGTGGTCTACCGCGAAAACACCGAGGATATCTACCTCGGCATCGAATGGAAGCAAGGTGATCCCACCGGCGA
TCGCCTGATCAAGCTGCTGAACGAGGACTTCATTCCCAACAGCCCCAGCTTGGGTAAAAAGCAAATCCGTTTGGAT
TCCGGCATTGGTATTAAGCCGATCAGTAAAACGGGTAGCCAGCGTCTGATTCGTCGTGCGATCGAGCATGCCCTAC
GCCTCGAAGGCCGCAAGCGACATGTCACCCTTGTCCACAAGGGCAACATCATGAAGTTCACGGAAGGTGCTTTCC
GGGACTGGGGCCTATGAACTGGCCACGACTGAGTTCCGAACCGACTGTGTGACTGAACGGGAGAGCTGGATTCTT
GCCAACCAAGAAAGCAAGCCGGATCTCAGCTTGGAAGACAATGCGCGGCTCATCGAACCTGGCTACGACGCGAT
GACGCCCGAAAAGCAGGCAGCAGTGGTGGCTGAAGTGAAAGCTGTGCTCGACAGCATCGGCGCCACCCACGGCA
ACGGTCAGTGGAAGTCTAAGGTGCTGGTTGACGATCGCATTGCTGACAGCATCTTCCAGCAGATTCAAACCCGCCC
GGGTGAATACTCGGTGCTGGCGACGATGAACCTCAATGGCGACTACATCTCTGATGCAGCGGCGGCGGTGGTCG
GTGGCCTGGGCATGGCCCCCGGTGCCAACATTGGCGACGAAGCGGCGATCTTTGAAGCGACCCACGGCACAGCG
CCCAAGCACGCTGGCCTCGATCGCATTAACCCCGGCTCGGTCATCCTCTCCGGCGTGATGATGCTGGAGTACCTAG
GCTGGCAAGAGGCTGCTGACTTGATCACCAAGGGCATCAGCCAAGCGATCGCTAACCGTGAGGTCACCTACGATC
TGGCTCGGTTGATGGAACCGGCGGTTGATCAACCACTCAAGTGCTCGGAATTTGCCGAAGCCATCGTCAAGCATTT
CGACGATTAGGGATCCAGCGC
SEQ ID NO. 5-
MAFFTAASKADFQHQLQAALAQHISEQALPQVALFAEQFFGIISLDELTQRRLSDLAGCTLSAWRLLERFDHAQPQVRV
YNPDYERHGWQSTHTAVEVLHHDLPFLVDSVRTELNRRGYSIHTLQTTVLSVRRGSKGELLEILPKGTTGEGVLHESLMY
LEIDRCANAAELNVLSKELEQVLGEVRVAVSDFEPMKAKVQEILTKLDNSAFAVDADEKNEIKSFLEWLVGNHFTFLGYE
EFTVVDQADGGHIEYDQNSFLGLTKMLRTGLTNEDRHIEDYAVKYLREPTLLSFAKAAHPSRVHRPAYPDYVSIREIDAD
GKVIKEHRFMGLYTSSVYGESVRVIPFIRRKVEEIERRSGFQAKAHLGKELAQVLEVLPRDDLFQTPVDELFSTVMSIVQIQ
ERNKIRVFLRKDPYGRFCYCLAYVPRDIYSTEVRQKIQQVLMERLKASDCEFWTFFSESVLARVQLILRVDPKNRIDIDPLQ
LENEVIQACRSWQDDYAALTVETFGEANGTNVLADFPKGFPAGYRERFAAHSAVVDMQHLLNLSEKKPLAMSFYQPL
ASGPRELHCKLYHADTPLALSDVLPILENLGLRVLGEFPYRLRHTNGREFWIHDFAFTAAEGLDLDIQQLNDTLQDAFVHI
VRGDAENDAFNRLVLTAGLPWRDVALLRAYARYLKQIRLGFDLGYIASTLNNHTDIARELTRLFKTRFYLARKLGSEDLDD
KQQRLEQAILTALDDVQVLNEDRILRRYLDLIKATLRTNFYQTDANGQNKSYFSFKFNPHLIPELPKPVPKFEIFVYSPRVE
GVHLRFGNVARGGLRWSDREEDFRTEVLGLVKAQQVKNSVIVPVGAKGGFLPRRLPLGGSRDEIAAEGIACYRIFISGLL
DITDNLKDGKLVPPANVVRHDDDDPYLVVAADKGTATFSDIANGIAIDYGFWLGDAFASGGSAGYDHKKMGITAKGA
WVGVQRHFRERGINVQEDSITVVGVGDMAGDVFGNGLLMSDKLQLVAAFNHLHIFIDPNPNPATSFAERQRMFDLP
RSAWSDYDTSIMSEGGGIFSRSAKSIAISPQMKERFDIQADKLTPTELLNALLKAPVDLLWNGGIGTYVKASTESHADVG
DKANDALRVNGNELRCKVVGEGGNLGMTQLGRVEFGLNGGGSNTDFIDNAGGVDCSDHEVNIKILLNEVVQAGDMT
DKQRNQLLASMTDEVGGLVLGNNYKQTQALSLAARRAYARIAEYKRLMSDLEGRGKLDRAIEFLPTEEQLAERVAEGH
GLTRPELSVLISYSKIDLKEQLLGSLVPDDDYLTRDMETAFPPTLVSKFSEAMRRHRLKREIVSTQIANDLVNHMGITFVQ
RLKESTGMTPANVAGAYVIVRDIFHLPHWFRQIEALDYQVSADVQLELMDELMRLGRRATRWFLRARRNEQNAARD
VAHFGPHLKELGLKLDELLSGEIRENWQERYQAYVAAGVPELLARMVAGTTHLYTLLPIIEAADVTGQDPAEVAKAYFA
VGSALDITWYISQISALPVENNWQALAREAFRDDVDWQQRAITIAVLQAGGGDSDVETRLALWMKQNDAMIERWR
AMLVEIRAASGTDYAMYAVANRELNDVALSGQAVVPAAATAELELA
SEQ ID NO. 6-
ATGGCGTTCTTCACLGCAGCCAGCAAAGCCGACTTCCAGCACCAACTGCAAGCGGCACTGGCGCAGCACATCAGT
GAACAGGCACTGCCACAAGTGGCGCTGTTCGCTGAACAATTCTTCGGCATCATTTCCCTGGACGAGCTGACCCAAC
GTCGCCTCTCCGACCTCGCTGGCTGTACTCTCTCTGCGTGGCGCCTGCTTGAGCGCTTCGATCACGCGCAACCGCA
AGTGCGCGTCTACAACCCCGATTACGAACGTCACGGCTGGCAGTCGACCCACACCGCGGTCGAAGTGCTGCACCA
CGACTTGCCGTTCCTGGTGGACTCGGTGCGTACCGAGCTGAACCGTCGCGGCTACAGCATCCACACCCTGCAGACC
ACCGTGCTGAGCGTGCGTCGTGGCAGCAAGGGCGAATTGCTGGAAATCCTGCCAAAAGGCACCACCGGCGAAGG
CGTTCTGCACGAGTCGCTGATGTACCTGGAAATCGACCGCTGCGCCAATGCGGCCGAATTGAATGTGCTGTCCAA
GGAACTGGAGCAGGTCCTGGGTGAAGTCCGCGTCGCGGTCTCCGATTTCGAGCCGATGAAGGCCAAGGTGCAGG
AAATCCTCACCAAGCTCGATAACAGCGCATTCGCCGTCGATGCCGACGAAAAGAATGAAATCAAGAGCTTCCTGG
AATGGCTGGTGGGCAACCACTTCACCTTCCTCGGCTACGAAGAGTTCACCGTTGTCGATCAGGCCGATGGCGGCC
ACATCGAATACGACCAGAACTCCTTCCTCGGCCTGACCAAGATGCTGCGCACCGGTCTGACCAACGAAGACCGCCA
CATCGAAGACTATGCCGTGAAGTACCTGCGCGAACCGACACTGCTGTCGTTCGCCAAGGCGGCGCATCCGAGCCG
CGTGCACCGTCCGGCCTACCCGGACTACGTGTCGATCCGCGAAATCGATGCCGACGGCAAAGTGATCAAGGAACA
CCGCTTCATGGGCCTGTACACCTCGTCGGTGTATGGCGAAAGCGTGCGTGTCATCCCGTTCATCCGCCGCAAGGTC
GAGGAAATCGAGCGTCGCTCCGGCTTCCAGGCCAAGGCTCACCTGGGCAAGGAACTGGCTCAGGTTCTGGAAGT
GCTGCCGCGTGACGATCTGTTCCAGACCCCGGTCGACGAACTGTTCAGCACCGTGATGTCGATCGTGCAGATCCA
GGAACGCAACAAGATCCGCGTGTTCCTGCGTAAAGACCCGTACGGTCGTTTCTGCTACTGCCTGGCCTACGTGCCG
CGTGACATCTACTCCACCGAAGTTCGCCAGAAGATCCAGCAAGTGCTGATGGAGCGCCTGAAAGCCTCCGACTGC
GAATTCTGGACGTTCTTCTCCGAGTCCGTGCTGGCCCGCGTGCAACTGATCTTGCGCGTCGACCCGAAAAACCGCA
TCGACATCGACCCGCTGCAACTGGAAAACGAAGTGATCCAGGCCTGCCGCAGCTGGCAGGACGACTACGCTGCCC
TGACCGTTGAAACCTTCGGCGAAGCCAACGGCACCAACGTGTTGGCCGACTTCCCGAAAGGCTTCCCGGCCGGCT
ACCGCGAGCGTTTCGCAGCGCATTCGGCCGTGGTCGACATGCAGCACTTGCTCAATCTGAGCGAGAAAAAGCCGC
TGGCCATGAGCTTTTACCAGCCGCTGGCCTCCGGCCCACGCGAGCTGCACTGCAAGCTGTATCACGCCGATACCCC
GCTGGCCCTGTCCGACGTGCTGCCGATCCTGGAAAACCTCGGCCTGCGCGTGCTGGGTGAGTTCCCGTACCGCCT
GCGTCATACCAACGGCCGCGAGTTCTGGATCCACGACTTCGCGTTCACCGCTGCCGAAGGCCTGGACCTGGACAT
CCAGCAACTCAACGACACCCTGCAGGACGCGTTCGTCCACATCGTCCGTGGCGATGCCGAAAACGATGCGTTCAA
CCGTCTGGTGCTGACCGCCGGCCTGCCATGGCGCGACGTGGCGCTGCTGCGTGCCTACGCCCGCTACCTGAAGCA
GATCCGCCTGGGCTTCGACCTCGGCTACATCGCCAGCACCCTGAACAACCACACCGACATCGCTCGCGAACTGACC
CGGTTGTTCAAGACCCGTTTCTACCTGGCCCGCAAGCTGGGCAGCGAGGATCTGGACGACAAGCAACAGCGTCTG
GAACAGGCCATCCTGACCGCGCTGGACGACGTTCAAGTCCTCAACGAAGACCGCATCCTGCGTCGTTACCTGGAC
CTGATCAAAGCAACCCTGCGCACCAACTTCTACCAGACCGACGCCAACGGCCAGAACAAGTCGTACTTCAGCTTCA
AGTTCAACCCGCACTTGATTCCTGAACTGCCGAAACCGGTGCCGAAGTTCGAAATCTTCGTTTACTCGCCACGCGTC
GAAGGCGTGCACCTGCGCTTCGGCAACGTTGCTCGTGGTGGTCTGCGCTGGTCGGACCGTGAAGAAGACTTCCGT
ACCGAAGTCCTCGGCCTGGTAAAAGCCCAGCAAGTGAAGAACTCGGTCATCGTGCCGGTGGGGGCGAAGGGCGG
CTTCCTGCCGCGTCGCCTGCCACTGGGCGGCAGCCGTGACGAGATCGCGGCCGAGGGCATCGCCTGCTACCGCAT
CTTCATTTCGGGCCTGTTGGACATCACCGACAACCTGAAAGACGGCAAACTGGTACCGCCGGCCAACGTCGTGCG
GCATGACGACGATGACCCGTACCTGGTGGTCGCGGCGGACAAGGGCACTGCAACCTTCTCCGACATCGCCAACGG
CATCGCCATCGACTACGGCTTCTGGCTGGGTGACGCGTTCGCGTCCGGTGGTTCGGCCGGTTACGACCACAAGAA
AATGGGCATCACCGCCAAGGGCGCGTGGGTCGGCGTACAGCGCCACTTCCGCGAGCGCGGCATCAATGTCCAGG
AAGACAGCATCACGGTGGTCGGCGTGGGCGACATGGCCGGTGACGTGTTCGGTAACGGCCTGTTGATGTCTGAC
AAGCTGCAACTGGTTGCTGCGTTCAACCACCTGCACATCTTCATCGACCCGAACCCGAACCCGGCCACCAGCTTCG
CCGAGCGTCAGCGCATGTTCGATCTGCCGCGCTCGGCCTGGTCCGACTACGACACCAGCATCATGTCCGAAGGCG
GCGGCATCTTCTCGCGCAGCGCGAAGAGCATCGCCATCTCGCCACAGATGAAAGAGCGCTTCGACATCCAGGCCG
ACAAGCTGACCCCGACCGAACTGCTGAACGCCTTGCTCAAGGCGCCGGTGGATCTGCTGTGGAACGGCGGTATCG
GTACCTACGTCAAAGCCAGCACCGAAAGTCACGCCGATGTCGGCGACAAGGCCAACGATGCGCTGCGCGTGAAC
GGCAACGAACTGCGCTGCAAAGTGGTGGGCGAGGGCGGTAACCTCGGCATGACCCAATTGGGTCGTGTGGAGTT
CGGTCTCAATGGCGGCGGTTCCAACACCGACTTCATCGACAACGCCGGTGGCGTGGACTGCTCCGACCACGAAGT
GAACATCAAGATCCTGCTGAACGAAGTGGTTCAGGCCGGCGACATGACCGACAAGCAACGCAACCAGTTGCTGGC
GAGCATGACCGACGAAGTCGGTGGTCTGGTGCTGGGCAACAACTACAAGCAGACTCAGGCCCTGTCCCTGGCGG
CCCGCCGTGCTTATGCGCGGATCGCCGAGTACAAGCGTCTGATGAGCGACCTGGAGGGCCGTGGCAAGCTGGAT
CGCGCCATCGAGTTCCTGCCGACCGAAGAGCAACTGGCCGAACGCGTTGCCGAAGGCCATGGCCTGACCCGTCCT
GAGCTGTCGGTGCTGATCTCGTACAGCAAGATCGACCTCAAGGAGCAGCTGCTGGGCTCCCTGGTGCCGGACGAC
GACTACCTGACCCGCGACATGGAAACGGCGTTCCCGCCGACCCTGGTCAGCAAGTTCTCCGAAGCTATGCGTCGTC
ACCGCCTCAAGCGCGAGATCGTCAGCACCCAGATCGCCAACGATCTGGTCAACCACATGGGCATCACCTTCGTTCA
GCGACTCAAAGAGTCCACGGGCATGACCCCGGCGAATGTTGCCGGTGCGTATGTGATTGTTCGGGATATCTTCCA
CCTCCCGCACTGGTTCCGTCAGATCGAAGCGCTGGACTACCAGGTTTCCGCTGACGTGCAGCTGGAGCTGATGGA
CGAGCTGATGCGTCTGGGCCGTCGCGCTACGCGCTGGTTCCTGCGTGCCCGTCGCAACGAGCAGAACGCTGCCCG
TGACGTCGCGCATTTCGGTCCGCACCTCAAAGAGCTGGGCCTGAAGCTGGACGAGCTGCTGAGCGGCGAAATCCG
CGAAAACTGGCAAGAGCGTTATCAGGCGTACGTCGCCGCCGGTGTTCCGGAACTGCTGGCGCGTATGGTGGCGG
GGACGACCCACCTCTACACGCTGCTGCCGATCATCGAAGCGGCCGACGTGACCGGCCAGGATCCAGCCGAAGTG
GCCAAGGCGTACTTCGCCGTGGGCAGCGCGCTGGACATCACCTGGTACATCTCGCAGATCAGCGCCTTGCCGGTT
GAAAACAACTGGCAGGCCCTGGCCCGTGAAGCGTTCCGCGACGACGTCGACTGGCAGCAACGCGCGATTACCATC
GCCGTTCTGCAAGCGGGTGGCGGTGATTCGGACGTGGAAACCCGTCTGGCACTGTGGATGAAGCAGAACGACGC
CATGATCGAACGCTGGCGCGCCATGCTGGTGGAAATCCGTGCCGCCAGCGGCACCGACTACGCCATGTACGCGGT
GGCCAACCGCGAGCTGAACGACGTGGCGCTGAGCGGTCAGGCAGTTGTGCCTGCTGCGGCGACTGCGGAGCTTG
AGCTTGCTTGA
SEQ ID NO. 7-
MIHAPSRWGVFPSLGLCSPDVVWNEHPSLYMDKEETSMTNLQTFELPTEVTGCAADISLGRALIQAWQKDGIFQIKTD
SEQDRKTQEAMAASKQFCKEPLTFKSSCVSDLTYSGYVASGEEVTAGKPDFPEIFTVCKDLSVGDQRVKAGWPCHGPV
PWPNNTYQKSMKTFMEELGLAGERLLKLTALGFELPINTFTDLTRDGWHHMRVLRFPPQTSTLSRGIGAHTDYGLLVIA
AQDDVGGLYIRPPVEGEKRNRNWLPGESSAGMFEHDEPWTFVTPTPGVWTVFPGDILQFMTGGQLLSTPHKVKLNT
RERFACAYFHEPNFEASAYPLFEPSANERIHYGEHFTNMFMRCYPDRITTQRINKENRLAHLEDLKKYSDTRATGS
SEQ ID. NO. 8-
ATGATACACGCTCCAAGTAGATGGGGAGTATTTCCCTCACTAGGGTTATGCAGCCCGGACGTTGTGTGGAATGAG
CATCCGAGCCTGTACATGGACAAAGAGGAAACCAGCATGACCAACCTGCAGACCTTTGAACTGCCGACCGAAGTG
ACCGGTTGCGCGGCGGACATCAGCCTGGGTCGTGCGCTGATTCAGGCGTGGCAAAAGGATGGTATCTTCCAGATT
AAAACCGACAGCGAGCAGGATCGTAAGACCCAAGAAGCGATGGCGGCGAGCAAGCAATTTTGCAAAGAGCCGCT
GACCTTCAAAAGCAGCTGCGTTAGCGACCTGACCTACAGCGGTTATGTGGCGAGCGGCGAGGAAGTTACCGCGG
GCAAGCCGGATTTCCCGGAAATTTTTACCGTGTGCAAGGACCTGAGCGTGGGCGATCAGCGTGTTAAAGCGGGTT
GGCCGTGCCATGGTCCGGTTCCGTGGCCGAACAACACCTATCAAAAGAGCATGAAAACCTTTATGGAGGAACTGG
GTCTGGCGGGCGAGCGTCTGCTGAAACTGACCGCGCTGGGTTTTGAACTGCCGATCAACACCTTCACCGACCTGA
CCCGTGATGGCTGGCACCACATGCGTGTGCTGCGTTTCCCGCCGCAGACCAGCACCCTGAGCCGTGGTATTGGTG
CGCACACCGACTACGGTCTGCTGGTGATTGCGGCGCAAGACGATGTTGGTGGCCTGTATATCCGTCCGCCGGTGG
AGGGCGAAAAGCGTAACCGTAACTGGCTGCCGGGCGAGAGCAGCGCGGGCATGTTTGAGCACGACGAACCGTG
GACCTTCGTTACCCCGACCCCGGGTGTGTGGACCGTTTTTCCGGGCGATATTCTGCAGTTCATGACCGGTGGCCAA
CTGCTGAGCACCCCGCACAAGGTTAAACTGAACACCCGTGAACGTTTCGCGTGCGCGTACTTTCACGAGCCGAACT
TCGAAGCGAGCGCGTATCCGCTGTTCGAGCCGAGCGCGAACGAACGTATCCACTACGGCGAGCACTTCACCAACA
TGTTTATGCGTTGCTATCCGGATCGTATCACCACCCAACGTATTAACAAAGAAAACCGTCTGGCGCACCTGGAAGA
CCTGAAGAAATACAGCGACACCCGTGCGACCGGCAGC
SEQ ID NO. 9-
MYKLVQTIVNSDEKNVLGDFILELGKDHKRYFLRNEILQAFADYCHQFPKPAYFYHSSSLGTFIQYTHEIILDGENTWFVV
RPKIASQEVWLLSADLTKFELMTPKALLDVSDRLVKRYQPHILEIDLHPFYSAAPRIDDSRNIGQGLTVLNHYFCNQALTD
PEYWIDALFQSLKRLEYNGIKLLISNHIHSGLQLTKQIKLALEFVSHLSPQTPYIKFKFHLQELGLEPGWGNNAARVRETLEL
LERLMDNPEPAILETFVSRICAVFRVVLISIHGWVAQEDVLGRDETLGQVIYVLEQARSLENKMRAEIKLAGLDTLGIKPHI
IILTRLIPNCEGTFCNLPLEKVDGTENAWILRVPFAESRPEITNNWISKFEIWPYLEKFALDAEAELLKQFQGKPNLIIGNYS
DGNLVAFILSRKMKVTQCNIAHSLEKPKYLFSNLYWQDLEAQYHFSAQFTADLISMNAADFIITSSYQEIVGTPDTMGQY
ESYKCFTMPNLYHVIDGIDLFSPKFNVVLPGVSENIFFPYNQTTNRESHRRQHIQDLIFHQEHPEILGKLDHPHKKPIFSVS
PITSIKNLTGLVECFGKSEELQKHSNLILLTSKLHPDLGTNSEEIQEIAKIHAIIDQYHLHHKIRWLGMRLPLRDIAETYRVIA
DFQGIYIHFALYESFSRSILEAMISGLPTFTTQFGGSLEIIENHDQGFNLNPTDLAGTAKTIINFLEKCENYPEHWLENSQW
MIERIRHKYNWNSHTNQLLLLTKMFSFWNFIYPEDNEARDRYMESLFHLLYKPIADHILSEHLSKIRNHN
SEQ ID NO. 10:-
ATGTATAAATTAGTGCAAACTATTGTTAACAGTGATGAAAAAAATGTTTTAGGTGACTTTATCTTAGAATTAGGCA
AGGATCATAAACGTTACTTTTTAAGAAATGAGATTTTACAAGCTTTTGCAGATTATTGTCACCAATTCCCAAAACCC
GCTTATTTTTATCACTCTTCCTCTTTAGGGACATCATTCAATACACCCATGAAATAATTTTAGATGGTGAAAATACT
TGGTTTGTAGTTAGACCAAAGATTGCGAGTCAAGAAGTATGGTTATTAAGCGCGGACTTGACTAAGTTTGAGTTAA
TGACACCGAAAGCATTATTAGATGTGAGCGATCGCTTAGTAAAGCGTTATCAACCGCACATTTTAGAAATTGATCT
CCATCCCTTTTATTCAGCAGCACCAAGAATTGATGATTCCAGAAATATTGGCCAAGGTTTAACCGTTCTTAATCATT
ATTTTTGTAATCAAGCATTGACAGATCCTGAATATTGGATTGACGCATTATTTCAATCATTAAAAAGATTAGAATAT
AACGGCATCAAATTATTAATTAGTAATCATATTCATTCAGGTTTGCAACTAACAAAGCAAATCAAACTAGCGTTAGA
ATTTGTGAGTCATTTATCCCCCCAGACACCATATATAAAATTTAAATTTCATCTTCAAGAACTCGGTTTAGAACCAG
GTTGGGGTAATAATGCAGCCAGAGTCAGAGAAACCTTAGAACTGCTGGAAAGACTCATGGATAATCCCGAACCTG
CAATTTTAGAAACCTTTGTTTCTCGCATTTGTGCAGTTTTCCGCGTCGTCCTTATTTCCATCCATGGTTGGGTTGCAC
AAGAAGATGTTTTAGGCAGAGATGAAACATTAGGACAAGTTATTTATGTTTTAGAACAAGCCCGCAGTTTAGAAA
ATAAAATGCGGGCAGAAATTAAACTTGCAGGTTTAGATACATTAGGAATTAAACCCCATATCATTATATTAACTCG
ACTGATTCCCAATTGTGAAGGCACATTTTGTAACTTACCATTAGAAAAAGTTGATGGTACAGAAAATGCTTGGATT
TTGCGCGTTCCTTTTGCAGAATCTCGACCGGAAATTACCAACAACTGGATTTCTAAATTTGAAATTTGGCCTTATTTA
GAAAAATTTGCTCTTGATGCCGAAGCAGAACTTTTAAAACAATTCCAAGGAAAGCCCAATCTAATTATTGGTAACT
ACAGTGACGGGAACTTAGTTGCTTTTATTCTCTCCCGAAAAATGAAAGTTACCCAATGTAATATTGCCCATTCCCTC
GAAAAACCTAAATATCTATTTAGTAACTTATATTGGCAAGATTTAGAAGCACAATATCACTTTTCTGCCCAATTTACC
GCTGATTTAATCAGTATGAATGCCGCAGATTTTATTATCACATCATCCTATCAAGAAATTGTAGGTACACCAGATAC
AATGGGACAATATGAATCTTATAAATGTTTCACCATGCCCAACTTATATCATGTAATTGATGGCATTGATTTATTTA
GCCCTAAATTCAATGTGGTATTACCAGGAGTCAGTGAAAATATATTTTTTCCCTACAACCAAACAACAAATAGAGA
ATCCCACCGTCGTCAACATATCCAAGACCTAATTTTCCATCAAGAACACCCAGAAATTCTCGGTAAATTAGATCATC
CTCATAAAAAACCGATCTTTTCCGTTAGTCCCATTACCTCAATTAAAAACCTCACAGGTTTAGTTGAATGTTTCGGTA
AAAGTGAAGAATTACAAAAACATAGTAACCTAATTTTATTAACCAGTAAACTTCATCCAGACTTAGGAACAAACTC
CGAAGAAATTCAAGAAATAGCAAAAATTCATGCGATTATTGATCAATATCATCTTCACCATAAAATCCGCTGGTTG
GGAATGCGTCTTCCTCTCCGCGATATTGCTGAAACCTATCGTGTAATTGCCGATTTTCAAGGGATTTATATTCACTTT
GCCCTTTATGAATCCTTTAGCAGAAGTATTTTAGAAGCAATGATTTCTGGATTACCAACTTTTACAACTCAATTTGGT
GGTTCATTAGAAATTATTGAAAACCATGATCAAGGATTTAACCTCAACCCCACAGACTTAGCAGGAACAGCCAAAA
CAATTATCAACTTCTTAGAAAAATGTGAAAATTATCCAGAACATTGGCTAGAAAATTCTCAATGGATGATTGAACG
CATTCGCCATAAATATAACTGGAATTCCCACACAAATCAACTCCTGTTATTAACGAAAATGTTTAGCTTTTGGAACT
TCATCTATCCCGAAGATAACGAAGCCAGAGATCGTTACATGGAAAGTTTATTTCATCTTCTTTATAAACCTATAGCT
GACCATATTTTATCAGAACATCTAAGCAAAATCAGAAATCATAATTAA
SEQ ID NO. 11:-
MAAQNLYILHIQTHGLLRGQNLELGRDADTGGQTKYVLELAQAQAKSPQVQQVDIITRQITDPRVSVGYSQAIEPFAPK
GRIVRLPFGPKRYLRKELLWPHLYTFADAILQYLAQQKRTPTWIQAHYADAGQVGSLLSRWLNVPLIFTGHSLGRIKLKKL
LEQDWPLEEIEAQFNIQQRIDAEEMTLTHADWIVASTQQEVEEQYRVYDRYNPERKLVIPPGVDTDRFRFQPLGDRGV
VLQQELSRFLRDPEKPQILCLCRPAPRKNVPALVRAFGEHPWLRKKANLVLVLGSRQDINQMDRGSRQVFQEIFHLVDR
YDLYGSVAYPKQHQADDVPEFYRLAAHSGGVFVNPALTEPFGLTILEAGSCGVPVVATHDGGPQEILKHCDFGTLVDVS
RPANIATALATLLSDRDLWQCYHRNGIEKVPAHYSWDQHVNTLFERMETVALPRRRAVSFVRSRKRLIDAKRLVVSDID
NTLLGDRQGLENLMTYLDQYRDHFAFGIATGRRLDSAQEVLKEWGVPSPNFWVTSVGSEIHYGTDAEPDISWEKHINR
NWNPQRIRAVMAQLPFLELQPEEDQTPFKVSFFVRDRHETVLREVRQHLRRHRLRLKSIYSHQEFLDILPLAASKGDAIR
HLSLRWRIPLENILVAGDSGNDEEMLKGHNLGVVVGNYSPELEPLRSYERVYFAEGHYANGILEALKHYRFFEAIA
SEQ ID NO. 12:-
GTGGCAGCTCAAAATCTCTACATTCTGCACATTCAGACCCATGGTCTGCTGCGAGGGCAGAACTTGGAACTGGGG
CGAGATGCCGACACCGGCGGGCAGACCAAGTACGTCTTAGAACTGGCTCAAGCCCAAGCTAAATCCCCACAAGTC
CAACAAGTCGACATCATCACCCGCCAAATCACCGACCCCCGCGTCAGTGTTGGTTACAGTCAGGCGATCGAACCCT
TTGCGCCCAAAGGTCGGATTGTCCGTTTGCCTTTTGGCCCCAAACGCTACCTCCGTAAAGAGCTaCTTTGGCCCCAT
CTCTACACCTTTGCGGATGCAATTCTCCAATATCTGGCTCAGCAAAAGCGCACCCCGACTTGGATTCAGGCCCACTA
TGCTGATGCTGGCCAAGTGGGATCACTGCTGAGTCGCTGGTTGAATGTACCGCTAATTTTCACAGGGCATTCTCTG
GGGCGGATCAAGCTAAAAAAGCTGTTGGAGCAAGACTGGCCGCTTGAGGAAATTGAAGCGCAATTCAATATTCAA
CAGCGAATTGATGCGGAGGAGATGACGCTCACTCATGCTGACTGGATTGTCGCCAGCACTCAGCAGGAAGTGGA
GGAGCAATACCGCGTTTACGATCGCTACAACCCAGAGCGCAAGCTTGTCATTCCACCGGGTGTCGATACCGATCGC
TTCAGGTTTCAGCCCTTGGGCGATCGCGGTGTTGTTCTCCAACAGGAACTGAGCCGCTTTCTGCGCGACCCAGAAA
AACCTCAAATTCTCTGCCTCTGTCGCCCCGCACCTCGCAAAAATGTACCGGCGCTGGTGCGAGCCTTTGGCGAACA
TCCTTGGCTGCGCAAAAAAGCCAACCTTGTCTTAGTACTGGGCAGCCGCCAAGACATCAACCAGATGGATCGCGG
CAGTCGGCAGGTGTTCCAAGAGATTTTCCATCTGGTCGATCGCTACGACCTCTACGGCAGCGTCGCCTATCCCAAA
CAGCATCAGGCTGATGATGTGCCGGAGTTCTATCGCCTAGCGGCTCATTCCGGCGGGGTATTCGTCAATCCGGCG
CTGACCGAACCTTTTGGTTTGACAATTTTGGAGGCAGGAAGCTGCGGCGTGCCGGTGGTGGCAACCCATGATGGC
GGCCCCCAGGAAATTCTCAAACACTGTGATTTCGGCACTTTAGTTGATGTCAGCCGACCCGCTAATATCGCGACTG
CACTCGCCACCCTGCTGAGCGATCGCGATCTTTGGCAGTGCTATCACCGCAATGGCATTGAAAAAGTTCCCGCCCA
TTACAGCTGGGATCAACATGTCAATACCCTGTTTGAGCGCATGGAAACGGTGGCTTTGCCTCGTCGTCGTGCTGTC
AGTTTCGTACGGAGTCGCAAACGCTTGATTGATGCCAAACGCCTTGTCGTTAGTGACATCGACAACACACTGTTGG
GCGATCGTCAAGGACTCGAGAATTTAATGACCTATCTCGATCAGTATCGCGATCATTTTGCCTTTGGAATTGCCACG
GGGCGTCGCCTAGACTCTGCCCAAGAAGTCTTGAAAGAGTGGGGCGTTCCTTCGCCAAACTTCTGGGTGACTTCC
GTCGGCAGCGAGATTCACTATGGCACCGATGCTGAACCGGATATCAGCTGGGAAAAGCATATCAATCGCAACTGG
AATCCTCAGCGAATTCGGGCAGTAATGGCACAACTACCCTTTCTTGAACTGCAGCCGGAAGAGGATCAAACACCCT
TCAAAGTCAGCTTCTTTGTCCGCGATCGCCACGAGACTGTGCTGCGAGAAGTACGGCAACATCTTCGCCGCCATCG
CCTGCGGCTGAAGTCAATCTATTCCCATCAGGAGTTTCTTGACATTCTGCCGCTAGCTGCCTCGAAAGGGGATGCG
ATTCGCCACCTCTCACTCCGCTGGCGGATTCCTCTTGAGAACATTTTGGTGGCAGGCGATTCTGGTAACGATGAGG
AAATGCTCAAGGGCCATAATCTCGGCGTTGTAGTTGGCAATTACTCACCGGAATTGGAGCCACTGCGCAGCTACG
AGCGCGTCTATTTTGCTGAGGGCCACTATGCTAATGGCATTCTGGAAGCCTTAAAACACTATCGCTTTTTTGAGGC
GATCGCTTAA
SEQ ID NO. 13:-
CAATTGCCCTAAGACAGTTGTCGTCTTTCGAAGTCTAGTTAACATTAGGGGCGATTCTTTGTTTCCACTGAGTGGAA
GCAAACGGTATCAAGGTTGCAGGCAGACTCAAGGTCTAGATTGCTTCACAGCTTGTGTGGCTATATTTATTATCTTC
ATTATTGATGGTAGTTGTGGGTGGATTTAAGATGGAAAAGTAACAGATAAATGTCGTCTTTAAGGGCGATCTAGA
TCGTATCGTTTTTAATTCCTAGGTCGGCATTTATTAATCAACCTCGATACAATATTTTTTTGTAAAAACTTCTAGATA
AATGACTCAAGTCTCATTGAAAGTCTGGGGTGTTGCCTCCCCAGTCAATTCAAGATTACCAAGGCCTCGCATCGCC
TCTTCTATTTTGTTTGAAGGGGACCTAACGTGTTGCGCCAAGCTAGTTCTCGACAGAGCATCTCAAGAGCGCGTTG
CTCGCGGGGGGCAAACAGTTGGAGATCAGCCAGCTCTTGCAAGACTTGTTGGGTGAGGTGGCTGGCAAAGCTAC
CGGCATAGCGCAGTAAGAGACTGTAGTAGCGAAATTGCGGTTGGCCGTTGCAATCGCGGTGAAAGGCAGCAATT
TCTGCTTCGCTGAGGCAGTAGACAGGGGTATTGACCGGCACGATCGCGCGAGGAATGCCCTGTTCGCCGTAGCGA
TCGCCGCCCTCTGTTTCCGCTAAGCTGCCGATCAAAACCGTGGAGAGCAGCGTGCGGGTTTCATTGATTAAATCAG
GCGTGAATAGTGGGTCGGGCCCACTTGAAAGACGCGGCCCGTTGTTCAGAAAGAGGGGATTAAACAACTGCGAG
TTGTAGACCACTCCGATCGCCCAATGGCGATCGCCTTCCAAACGAACAAAGCTGCCAAATCCATAGCTCTCGGCGG
CTGGTGGATTGGAGACATCCATGTCGTCATCCACTTGGACAACGTAGTCACAGTGCGAGTTGGATTTGACAACTTT
GCCGAGGCGCATGGTGCTGCCAGTGTTACAACCAATTAACCAATTCTGACATATGGACACCATCGAATGGTGCAA
AACCTTTCGCGGTATGGCATGATAGCGCCCGGAAGAGAGTCAATTCAGGGTGGTGAATGTGAAACCAGTAACGTT
ATACGATGTCGCAGAGTATGCCGGTGTCTCTTATCAGACCGTTTCCCGCGTGGTGAACCAGGCCAGCCACGTTTCT
GCGAAAACGCGGGAAAAAGTGGAAGCGGCGATGGCGGAGCTGAATTACATTCCCAACCGCGTGGCACAACAACT
GGCGGGCAAACAGTCGTTGCTGATTGGCGTTGCCACCTCCAGTCTGGCCCTGCACGCGCCGTCGCAAATTGTCGC
GGCGATTAAATCTCGCGCCGATCAACTGGGTGCCAGCGTGGTGGTGTCGATGGTAGAACGAAGCGGCGTCGAAG
CCTGTAAAGCGGCGGTGCACAATCTTCTCGCGCAACGCGTCAGTGGGCTGATCATTAACTATCCGCTGGATGACCA
GGATGCCATTaCTGTGGAAGCTaCCTGCACTAATGTTCCGGCGTTATTTCTTGATGTCTCTGACCAGACACCCATCA
ACAGTATTATTTTCTCCCATGAAGACGGTACGCGACTGGGCGTGGAGCATCTGGTCGCATTGGGTCACCAGCAAAT
CGCGCTGTTAGCGGGCCCATTAAGTTCTGTCTCGGCGCGTCTGCGTCTGGCTGGCTGGCATAAATATCTCACTCGC
AATCAAATTCAGCCGATAGCGGAACGGGAAGGCGACTGGAGTGCCATGTCCGGTTTTCAACAAACCATGCAAATG
CTGAATGAGGGCATCGTTCCCACTGCGATGCTGGTTGCCAACGATCAGATGGCGCTGGGCGCAATGCGCGCCATT
ACCGAGTCCGGGCTGCGCGTTGGTGCGGATATCTCGGTAGTGGGATACGACGATACCGAAGACAGCTCATGTTAT
ATCCCGCCGTTAACCACCATCAAACAGGATTTTCGCCTGCTGGGGCAAACCAGCGTGGACCGCTTGCTGCAACTCT
CTCAGGGCCAGGCGGTGAAGGGCAATCAGCTGTTGCCCGTTTCACTGGTGAAAAGAAAAACCACCCTGGCGCCCA
ATACGCAAACCGCCTCTCCCCGCGCGTTGGCCGATTCATTAATGCAGCTGGCACGACAGGTTTCCCGACTGGAAAG
CGGGCAGTGAGCTTTGTTGACAATTAATCATCCGGCTCGTATAATGTGTGGAATTGTGAGCGGATAACAAGAAGG
AGATGAGTATTCAACATTTCCGTGTCGCCCTTATTCCCTTTTTTGCGGCATTTTGCCTTCCTGTTTTTGCTCACCCAGA
AACGCTGGTGAAAGTAAAAGATGCTGAAGATCAGTTGGGTGCACGAGTGGGTTACATCGAACTGGATCTCAACA
GCGGTAAGATCCTTGAGAGTTTTCGCCCCGAAGAACGTTTTCCAATGATGAGCACTTTTAAAGTTCTGCTATGTGG
CGCGGTATTATCCCGTATTGACGCCGGGCAAGAGCAACTCGGTCGCCGCATACACTATTCTCAGAATGACTTGGTT
GAGTACTCACCAGTCACAGAAAAGCATCTTACGGATGGCATGACAGTAAGAGAATTATGCAGTGCTGCCATAACC
ATGAGTGATAACACTGCGGCCAACTTACTTCTGACAACGATCGGAGGACCGAAGGAGCTAACCGCTTTTTTGCACA
ACATGGGGGATCATGTAACTCGCCTTGATCGTTGGGAACCGGAGCTGAATGAAGCCATACCAAACGACGAGCGT
GACACCACGATGCCTGTAGCAATGGCAACAACGTTGCGCAAACTATTAACTGGCGAACTACTTACTCTAGCTTCCC
GGCAACAATTAATAGACTGGATGGAGGCGGATAAAGTTGCAGGACCACTTCTGCGCTCGGCCCTTCCGGCTGGCT
GGTTTATTGCTGATAAATCTGGAGCCGGTGAGCGTGGGTCTCGCGGTATCATTGCAGCACTGGGGCCAGATGGTA
AGCCCTCCCGTATCGTAGTTATCTACACGACGGGGAGTCAGGCAACTATGGATGAACGAAATAGACAGATCGCTG
AGATAGGTaCCTCACTGATTAAGCATTGGTAATTGTTCAGAACGCTCGGTCTTGCACACCGGGCGTTTTTTCTTTGT
GAGTCCAGGTACCAATCAATCTCCCCCAAGTCAAGCGGCGCTGAGACCCAGTGTCTGCCGGTGAGTCAGTCTTGG
CAAGCAAACTGTGCCTTTGCGATTTCTTACCCTACGCAGCTCCGGGATCGATCGGAGGTAACCAAGGCTACGGACA
ATGGCGCGGGCACCAGCTGTTGGTAAACTGAGGAGCGATCGCCGCTTCAGTCCAAAGGCTATGACGCAAAAATCC
GTTGTTATTGCTCCGTCCATTCTGTCAGCGGATTTCAGCCGCTTGGGCGACGATGTCCGCGCTGTTGACCAGGCTG
GCGCTGACTGGATTCACGTCGATGTGATGGATGGTCGCTTCGTCCCTAACATCACCATTGGACCGCTGATCGTTGA
AGCGCTGCGCCCGGTGACCCAAAAGCCGTTGGACGTCCACTTGATGATCGTCGAGCCGGAAAAATATGTGCCGGA
TTTCGCGAAAGCAGGGGCTGACATCATCTCGGTCCAAGCAGAAGCTTGCCCCCACCTGCACCGCAACTTGGCTCAG
ATCAAAGACCTCGGCAAGCAAGCAGGCGTCGTCCTCAACCCCTCTACCCCAGTCGAAACCCTGGAATACGTGCTGG
AGTTGTGCGACCTGATTTTGATCATGAGCGTCAACCCTGGCTTCGGTGGTCAGAGTTTCATCCCAGCTGTCCTGCC
GAAAATCCGTAAGCTGCGCGCCATGTGCGATGAGCGTGGCCTTGATCCTTGGATTGAAGTCGATGGCGGCTTGAA
AGCCAATAACACTTGGCAGGTGCTGGAAGCCGGTGCTAACGCAATTGTGGCGGGCTCGGCAGTCTTCAACGCGCC
GGACTATGCTGAAGCGATCGCGGCGATTCGCAACAGCAAGCGTCCTGAACTTGTCACTGCTTAGGCTTCTCGCTCA
ACGCTCAGTGGAGCAATCTGAATCTTGCAGCCCTTCAGTGGATCAGTCTGCTGAGGGGTTTTGCTTTAGGATGGGC
GATCGCGAGTAGGGACACGGATCGCTGGTA

1. A process comprising:
(i) providing a gaseous stream including greater than 1% by volume carbon dioxide;
(ii) providing water;
(iii) converting the carbon dioxide and the water to an organic intermediate and oxygen gas in the presence of light;
(iv) separating the oxygen gas from the organic intermediate; and
(v) converting the organic intermediate to ethylene and carbon dioxide after said step of separating the oxygen gas from the organic intermediate.

2. The process of claim 1, where said step (iii) of converting the organic intermediate to ethylene and carbon dioxide takes place in the presence of excess water from said step (i) of converting carbon dioxide and water to an organic intermediate, and where said step of (iii) converting the organic intermediate to ethylene and carbon dioxide consumes only a portion of the organic intermediate formed in said step (i) of converting carbon dioxide and water to an organic intermediate and oxygen gas, and further comprising the step of routing the excess water and organic intermediate from said step (iii) of converting the organic intermediate to ethylene and carbon dioxide back to said step (i) of converting the carbon dioxide and water to an organic intermediate.

3. The process of claim 1, where said step (i) of converting carbon dioxide and water to an organic intermediate and oxygen gas takes place within a first bioreactor that contains a microorganism that produces an organic intermediate and oxygen gas from the carbon dioxide in the presence of light energy.

4. The process of claim 1, where said gaseous stream includes greater than 3% by volume carbon dioxide.

5. The process of claim 1, further comprising the step of compressing a flue gas stream from a combustion step to form the gaseous stream including carbon dioxide.

6. The process of claim 1, where said step (iv) of separating oxygen from the organic intermediate produces an organic intermediate-rich stream that is introduced to said step (v) of converting the organic intermediate to ethylene and carbon dioxide.

7. The process of claim 1, where said step of separating the oxygen forms an organic intermediate-rich stream that includes less than 30 ppm oxygen.

8. The process of claim 3, where said step of separating the oxygen gas includes venting oxygen gas from the first bioreactor.

9. The process of claim 1, where said step (v) of converting the organic intermediate to ethylene and carbon dioxide takes place within a second bioreactor that contains a microorganism that produces an enzyme for converting the organic intermediate to ethylene and carbon dioxide.

10. The process of claim 1, where said step (v) of converting the organic intermediate to ethylene and carbon dioxide takes place in the substantial absence of light energy.

11. The process of claim 1, where the ethylene and the carbon dioxide formed in said step (v) of converting the organic intermediate form a product stream.

12. The process of claim 11, further comprising the step of converting oxygen gas within the product stream to carbon dioxide.

13. The process of claim 11, further comprising the step of removing water from the product stream.

14. The process of claim 11, further comprising separating carbon dioxide from the product stream.

15. The process of claim 14, where said step of separating carbon dioxide from the product stream produces an ethylene-rich stream.

16. The process of claim 11, where said step of separating the carbon dioxide from the product stream produces a carbon dioxide stream, and where the carbon dioxide stream is introduced to a step (ii) of converting carbon dioxide to an organic intermediate.

17. The process of claim 1, where said step of converting carbon dioxide and water to an organic intermediate and oxygen gas takes place within a first bioreactor.

18. The process of claim 1, where said separating oxygen gas from the organic intermediate includes venting the oxygen gas from an aqueous medium in which the organic intermediate is contained.

19. The process of claim 1, where said step of converting the organic intermediate to ethylene and carbon dioxide after said step of separating the oxygen gas from the organic intermediate takes place within a second bioreactor.

20. A system for producing ethylene, the system comprising:
(i) a first bioreactor including photosynthetic microorganisms that convert carbon dioxide to an organic intermediate, said first bioreactor having a carbon dioxide inlet and an outlet for the organic intermediate; and
(ii) a second bioreactor in fluid communication with the first bioreactor and including microorganisms that convert the organic intermediate produced in the first bioreactor to ethylene, said second bioreactor having an outlet for gaseous materials including ethylene, and said second bioreactor having an outlet for fluid materials including unreacted organic intermediate.

21. The system of claim 20, where the outlet for fluid materials of said second bioreactor is in fluid communication with said first bioreactor.

22. The system of claim 20, where the outlet for gaseous materials of said second bioreactor is in fluid communication with a carbon dioxide separator.

23. The system of claim 22, where the carbon dioxide separator includes an outlet for purified carbon dioxide, and where said outlet for purified carbon dioxide is in fluid communication with the first bioreactor.
